(19) United States
(12) Patent Application Publication
(10) Pub. No.: US 2026/0341418 A1
(43) Pub. Date: Oct. 29, 2026

(54) METHOD FOR PLANNING AN EXPERIMENT SERIES

(71) Applicant: BASF SE, Ludwigshafen am Rhein (DE)

(72) Inventors: Volker Settels, Ludwigshafen am Rhein (DE); Andreas Buechse, Ludwigshafen am Rhein (DE); Astrid Niederle, Freeport, TX (US)

(21) Appl. No.: 19/103,619

(22) PCT Filed: Aug. 15, 2023
(86) PCT No.: PCT/EP2023/072607

§ 371 Date: Feb. 13, 2025

(30) Foreign Application Priority Data
Aug. 16, 2022 (EP) 22190496.4

(51) Int. Cl. G16C 20/10; G16C 20/70

(52) U.S. Cl. CPC G16C 20/10; G16C 20/70

(57) ABSTRACT

The invention refers to a method for providing test synthesis specifications for performing an experiment series. Synthesis parameters defining the ingredient and process parameter ranges in which test polymers for the experiment series are to be determined are provided. A plurality of potential test synthesis specifications are provided based on the synthesis parameters. Computed characterizing parameters are provided indicative of a computed characteristic of a polymer. A computed characterizing parameter value is determined for the provided computed characterizing parameters based on the plurality of potential test synthesis specifications. Test synthesis specifications are selected from the plurality of potential test synthesis specifications based on the determined computed characterizing parameter values and the test synthesis specifications are provided.

### FIELD OF THE INVENTION

The invention relates to a method, an apparatus and a computer program product for providing one or more test synthesis specifications each associated with a test polymer for performing an experiment series. Further, the invention refers to a method, apparatus and computer program product for generating a machine learning based determination model. Moreover, the invention refers to a method, apparatus and computer program product for automated performing of an experiment series for finding a target polymer comprising a predetermined target technical application property. Further, the invention refers to interface methods, interface apparatuses, and interface computer program products providing an interface for the above methods, apparatuses and computer program products.

### BACKGROUND OF THE INVENTION

Generally, polymers are widely used in industrial and/or daily use products due to their broad range of application properties. For developing new polymers or for utilizing known polymers in new application contexts, often polymers have to be found that comprise a specific technical application property, for instance, a specific heat insulating factor, a specific hardness, or a specific reflectivity. Today, the search for such new polymers is often performed by defining a search range for the polymers to be searched and then designing a plurality of experiments based on expert knowledge or a statistical design of experiment (DoE) in order to successively try to find within the plurality of experiments a polymer that fulfils the respectively searched property. However, the number of experiments, necessary to find a respective target polymer, be very large, for example, it can be necessary to synthesize a few hundred polymers during the search. Particularly, if many different chemical substances and process conditions can be varied, statistical design of experiment results in a vast number of necessary experiments. Further the process is mainly based on the experience, instincts and knowledge of the experts designing the experiment series. Thus, the success of such an experiment series is often difficult to predict and a lot of resources can be wasted during the experimental search process. Thus, it would be advantageous if a method could be found that allows for a more objective and efficient selection of the respective experiments of the experiment series, in particular, that allows to reduce the number of experiments that have to be performed.

### SUMMARY OF THE INVENTION

It is an object of the present invention to provide methods, apparatuses and computer program products that allow to perform an experiment series more objectively and more efficiently, i.e. with less performed experiments and thus with less resources, preferably, for applications referring to a determining of a training dataset for training a polymer property determination model or a finding of a target polymer comprising a target value for a target technical application property.

In a first aspect of the present invention, a computer implemented method for providing one or more test synthesis specifications each associated with a test polymer for performing of an experiment series is presented, wherein a test synthesis specification is suitable for synthesising the respective test polymer and measuring one or more technical application properties of the test polymer in the experiment series, wherein the method comprises a) receiving synthesis parameters defining the ingredient and/or process parameter ranges in which one or more test polymers for the experiment series are to be determined, wherein different ingredient and/or process parameters are associated with different potential test polymers, b) deriving a plurality of potential test synthesis specifications, which, for example, are composed out of synthesis parameters, associated with respective potential test polymers based on the synthesis parameters, c) providing computed characterizing parameters, wherein a computed characterizing parameter is indicative of a computed characteristic of a polymer and/or is derivable from one or more computed characteristics of the polymer, d) determining for the provided computed characterizing parameters a computed characterizing parameter value for the potential test polymers based on the plurality of potential test synthesis specifications, e) determining, in particular, selecting, one or more test synthesis specifications associated with respective test polymers from the plurality of potential test synthesis specifications based on the determined computed characterizing parameter values, f) providing the one or more test synthesis specifications comprising instructions for controlling a synthesis of the respective one or more test polymers.

Since the one or more test synthesis specifications associated with respective test polymers are selected from the plurality of potential test synthesis specifications based on the determined computed characterizing parameter values, the test polymers to be utilized during the experiment series can be determined based on an objective criterion. Moreover, the determined computed characterizing parameter values can also be utilized to only determine test polymers from the plurality of potential test polymers for which the determined computed characterizing parameter values indicate a respective success for the experiment series. Thus, not only the amount of to be performed experiments, i.e. the amount of test polymers, can be reduced, but also the respective test polymers can be selected according to an objective criterion. Thus, the method allows for a more objective and efficient performing of an experiment series in particular for applications like determining a training dataset for a property determination model or finding a target polymer with a respective target technical application property.

The method refers to a computer implemented method and thus can be performed by a general or dedicated computer, or network of computers, adapted to perform the method, for instance, by executing a respective computer program. The method is configured for providing one or more test synthesis specifications each associated with a test polymer for performing of an experiment series. Generally, a synthesis specification is defined as an instruction on how a polymer can be synthesized. In particular, the synthesis specification indicates the starting ingredients and the respective parameters for polymerization, like an amount of ingredients and feed profiles for the ingredients, and thus comprises the synthesis parameters defining the process and/or ingredient parameters for a specific polymer. The process parameters can also cover all aspects regarding the apparatus used for polymerization, like a temperature profile, a reactor type and size, or stirring power. The performing of the experimental series can comprise the planning, actual synthesising and experimenting, and the processing of the measurement results. The experiment series can refer to any experiment series comprising one or more objectives with respect to a polymer or a material comprising a polymer. Preferably, it is an object of the experiment series to determine a training dataset that allows to efficiently train a property determination model that can determine a technical application property of a polymer based on one or more computed characteristics of the polymer after the training. A further preferred object of the experiment series refers to finding a target polymer that comprises the predetermined target value for a target technical application property, for instance, in the context of developing a new polymer or finding existing polymers for specific applications. However, the experiment series can also refer to any other objective associated with measuring one or more technical application properties of a polymer. Preferably, the experiment series refers to a design of experiment (DoE) process and the test polymers provided by the method refer to the experiments in the DoE process.

A technical application property can generally refer to any property of a polymer and/or a substance consisting at least partly of the polymer, for example, a formulation or mixture comprising the polymer, that allows to assess a technical applicability of the respective polymer as provided after its synthesis. Preferably, the technical application property comprises at least one of mechanical properties, optical properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. An optical property can generally comprise any of coloration, turbidity, opaqueness, lucidity, reflection, appearance, absorption, scattering, color strength, colour hue, colour saturation, colour intensity, cloud point, matting degree, optical density, spectra, refractive index. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, and charge density. The chemical property can comprise any of chemical resistance, reaction timing, demolding time, growing, hard/soft segment content, crystallinity, reaction temperature, reaction pressure, decomposition, thermal decomposition, photodegradation, acidity, pKa, pH, moisture/water content, flammability, burning rate, selfignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, in particular, resistance against a pathogenic virus, bacterium, fungus, plant or animal or developmental stage of said pathogen, tolerance against environ-mental parameters, e.g. drought tolerance, resistance against enzymatic degradation, e.g. protease resistance, lipase resistance, amylase resistance, hydrolase resistance, pesticide resistance, toxicity, biotransformation, ecotoxicology, sensitization, in particular, allergenicity, bacterial count, enzyme activity, substrate specificity, cofactor dependence, product specificity, substrate and/or product inhibition, dissociation constant, Michaelis-Menten-kinetics values, activity/stability at or in different: pH, temperature, pressure, organic solvent concentration, carrier formulations, encapsulation formulations; distribution in environment, compartmentalization, bioaccumulation, biological exposure LD50, mutagenicity.

In a first step, the method comprises receiving synthesis parameters defining the ingredient and/or process parameter ranges in which one or more test polymers for the experiment series are to be determined. In particular, synthesis parameters can refer to any parameter that allows to define a search space in which the respective experiment series should be performed, for example, can comprise ingredient and/or process parameters at the end of the respective range. The ingredient parameters generally quantify the production of the polymer itself, in particular, the substances utilized for producing the polymer. Such substances can refer to starting substances like initiators, monomers, or prepolymers from which the polymer is produced. However, the substances can also refer to auxiliary substances, like catalysts or surfactants. The substances can refer to mixtures of different ingredients as well, like catalysts dissolved in a solvent. In this context, the ingredient parameters quantify the influence of these substances on the produced polymer and thus also characterize the polymer itself. For example, the ingredient parameters can refer to any of an amount and/or feet profile of specific monomers, an amount and/or feed profile of specific additives, a mixing ration between different substances, or a used solvent. The process parameters can, in this context, refer to variables that can be set during the synthesis of a test polymer. Thus, process parameters quantify the production process of the respective polymer and can be part of a synthesis specification. For example, the process parameters can refer to any of a temperature profile, a pressure, a stirring power, a reactor type, etc. The ingredient parameters generally quantify the production of the polymer itself, in particular, the substances utilized for producing the polymer. Such substances can refer to starting substances like prepolymers from which the polymer is produced. However, the substances can also refer to auxiliary substances, like catalysts. In this context, the ingredient parameters quantify the influence of these substances on the produced polymer and thus also characterize the polymer itself. For example, the ingredient parameters can refer to any of an amount of specific monomers, an amount of specific additives, a mixing ration between different substance, or a used solvent.

Thus, the received synthesis parameters define ingredient and process parameter ranges for which test polymers for the experiment series can be determined. In particular, it is preferred that the synthesis parameters take constraints for these parameters already into account. For example, if an automated synthesis apparatus that is configured for synthesizing polymers based on a synthesis specification only provides a respective temperature range during the synthesis process, the process parameters can be received such that this temperature range is already taken into account. However, the synthesis parameters can also be provided independent of such constraints and possible constraints can also be taken into account in later steps of the method. Generally, a variation of any of the ingredient and/or process parameters is also associated with a different polymer. Thus, different ingredient and/or process parameters are associated with different potential test polymers such that the ingredient and/or process parameter ranges also define the search space for the test polymers.

In a next step, a plurality of potential test synthesis specifications associated with respective potential test polymers are derived based on the synthesis parameters and thus based on the ingredient and/or process parameter ranges. In particular, the plurality of potential test synthesis specifications are derived such that they fall within the ingredient and/or process parameter ranges. Generally, the potential test synthesis specifications can be derived and provided, for example, by an expert. However, the plurality of potential test synthesis specifications can also automatically be derived and provided, for example, by selecting from a plurality of potential test synthesis specifications already generated and, for example, stored on a respective storage, the plurality of potential test synthesis specifications that fall within the ingredient and/or process parameter ranges. Moreover, the deriving of the plurality of potential test synthesis specifications can also refer to a generating of the plurality of potential test synthesis specifications or at least a part of the plurality of the potential test synthesis specifications. For example, potential test synthesis specifications can be generated by varying from a starting synthesis specification one or more parameters of the starting synthesis specification, for instance, process parameters or ingredient parameters, while taking the ingredient and/or process parameter ranges into account or by selecting after the generations the potential test synthesis specifications that fall within the ingredient and/or process parameter ranges. The generating of the potential test synthesis specification can be performed in an arbitrary manner, for instance, by arbitrarily varying one or more parameters of the potential test synthesis specification or can be generated in accordance with predetermined rules, for instance, based on a predetermined scheme for the variation of the parameters. In particular, it is preferred that the plurality of potential test synthesis specifications is derived such that it covers the ingredient and/or process parameter ranges in accordance with a predetermined criterion. For example, such a criterion can refer to a predetermined distribution of the plurality of potential test synthesis specifications over the ingredient and/or process parameter ranges. In particular, statistical criterions, like defined average distances between potential test synthesis specifications, a number of potential test synthesis specifications, etc. can be utilized as respective criterion. However, the plurality of potential test synthesis specifications can also be derived without utilizing such a criterion, wherein in this case, it is preferred that a respective larger amount of potential test synthesis specifications is derived. For example, the number of potential test synthesis specifications for the plurality of potential test synthesis specifications can be predetermined based on the size of an ingredient and/or process parameter range. Since generally, the deriving of a plurality of potential test synthesis specifications is an easy and resource-preserving task, deriving a huge amount of potential test synthesis specifications provides no disadvantages.

In a next step, computed characterizing parameters are provided. Generally, the computed characterizing parameters can be any computed characterizing parameters. Preferably, a plurality of computed characterizing parameters are provide. In particular, it is preferred that the amount of provided computer characterizing parameters is statistically selected to increase the chances that at least some of the provided characterizing parameters are highly relevant for determination of the technical application property. However, the selection of the test synthesis specifications is generally independent on whether or not the provided computed characterizing parameters have an influence on the technical application property. For example, polymers with similar computed characterizing parameters that do not have an influence on the technical application property can also be regarded as being indicative of these polymers having also similar computed characterizing parameters. Although this assumption might not be true for all cases, it still allows for a suitable accuracy when determining the test synthesis specifications. However, the accuracy and efficiency of the method can be increased if at least some of the provided selected application properties are associated with the technical application property. Thus, preferably, the provided computed characterizing parameters are associated with the one or more technical application properties. Generally, a computed characterizing parameter is associated with the technical application property if it is expected, for instance, due to theoretical or experimental considerations, that the computed characterizing parameter has a direct or indirect influence on the technical application property of a polymer. Generally, the computed characterizing parameters are parameters that are determined based on computer calculations or simulations and characterize and/or quantify one or more characteristics of the polymer.

The computed characterizing parameters can comprise, preferably, physicochemical parameters, for instance, computed physicochemical polymer properties, that are indicative of physicochemical characteristics of the polymer. In particular, the polymer physicochemical parameters are indicative of parameters quantifying the physicochemical characteristics of the polymer. In this context, the term “physicochemical characteristics” refers to physical and/or chemical characteristics of the polymer. For example, the physicochemical parameter can refer to any of a molar mass distribution, a viscosity, a degree of protonation, etc. However, additionally or alternatively to the physicochemical parameters the computed characterizing parameters can also comprise parameters referring to subgroups of a polymer, for example, parameters indicating types and amounts of subgroups in a polymer.

In a preferred embodiment, the computed characterizing parameters are indicative of the types and amounts of the subgroups of the polymer. Moreover, the computed characterizing parameters can comprise parameters quantifying the subgroups of the polymer and amount of auxiliary substances. In these embodiments, the computed characterizing parameters can be derived by determining subgroups of the polymer. Generally, a subgroup refers to a part of the polymer, wherein all subgroups of a polymer together form the polymer. For example, a subgroup can refer to a part of the polymer, wherein the subgroups are linked together successively along a chain or network to form the polymer. Preferably, the subgroups of the polymer refer to repeating units that describe a part of the polymer which when repeated produces the complete polymer chain. However, in some cases, a subgroup can also refer to a single part of the polymer that is not repeated. Moreover, it is preferred that the subgroups comprise parts that are repeated, for example, a subgroup of a polymer can comprise a repeating core also present in other subgroups and further additional parts that are not present in other subgroups. Preferably, the subgroups refer to at least one of polymerized monomers or oligomer fragments. More preferably, the subgroups refer to polymerized monomers. In this context, polymerized monomers refer to monomers after their polymerization sometimes also called “mer unit” or “mer”. In particular, polymerized monomers do not refer to monomers, i.e. raw materials, as present in a reaction mixture before polymerization, but refer to repeating units derived from monomers that have been changed during or after the polymerization. Thus, subgroup computed characterizing parameters determined for polymerized monomers are different from subgroup computed characterizing parameters determined for unreacted monomers before polymerization. It has been found by the inventors that in particular the polymerized monomers allow to determine polymer computed characterizing parameters from the subgroup computed characterizing parameters of the polymerized monomers that allow to accurately characterize a polymer. In a preferred embodiment, method comprises determining, from the potential test synthesis specifications of the potential test polymers, the subgroups of the respective test polymer, for instance, to determine a chemical structure of the subgroups after their polymerization. Even more preferably, a molecular model of a subgroup is determined in a way that is suited for quantum chemical computations regarding a number of atoms and their connectivity that is representative of the properties of the subgroup within the polymer. Moreover, additionally or alternatively to a molecular model of a subgroup treating the subgroup as a monomer structure, also a molecular model referring to an oligomer model can be utilized that takes into account effects of neighbouring molecular structures of the subgroup in the polymer.

In a further step computed characterizing parameter values of the computed characterizing parameters are determined from the potential test synthesis specifications. If the computed characterizing parameters are based on computed characterizing parameters of subgroups or refer to parameters indicative of the subgroups, it is preferred that the computed characterizing parameters are determined by first determining the subgroups of the polymer. For example, respective subgroups of the polymer can be determined utilizing known methods. In particular, it is preferred that the subgroups are determined such that between atoms of different subgroups in the polymer the bond is as least polarized as possible and, preferably, with a bond order as small as possible (e.g. a CC single bond). Additionally, it is preferred that the subgroups representing a polymer comprise the same number of active non-hydrogen-atoms then the polymer. Besides the active atoms, a subgroup can also contain further atoms, which can be ignored during computing the computed characterizing parameters of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languishes like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of monomers of the polymer are directly derivable and, for example, RDkit can be used to determine from the SMILES of the monomers the SMILES, i.e. the number and connectivity of the atoms, of the subgroups.

The determined subgroups of the polymer are associated with subgroup computed characterizing parameters indicative of parameters quantifying, in particular, physicochemical characteristics of the subgroups in the polymer. In this case, it is preferred that the computed characterizing parameters are determined by determining respective subgroup computed characterizing parameters for each of the subgroups and to determine the computed characterizing parameters based on the subgroup computed characterizing parameters of the subgroups, for instance, by averaging. Thus, in this embodiment the method preferably comprises first providing or determining for a respective potential test polymer the subgroups from the corresponding potential test synthesis specification, then to determine or provide the subgroup computed characterizing parameters, i.e. values of the parameters quantifying the computed characterizing parameters, of the subgroups, and then to determine the polymer computed characterizing parameters based on the subgroup computed characterizing parameters of each polymer. However, the determined subgroups can also be utilized to determine subgroup parameters as computed characterizing parameters, for example, the presents or absents of specific subgroups, an amount of a type of subgroup, relations between the amount of specific subgroups, etc.

Preferably, the polymer computed characterizing parameters comprise physicochemical parameters referring to at least one of constitutional descriptors, count descriptors, list of structural fragments, fingerprints, graph invariants, 3D-descriptors and/or higher dimensional descriptors that are indicative of parameters quantifying physicochemical characteristics of the polymer. Moreover, it is preferred that the polymer computed characterizing parameter comprise as physicochemical parameter a molar mass distribution of the polymer. In a preferred embodiment the polymer computed characterizing parameters comprise 3D descriptors, in particular quantum chemical descriptors. Generally, the polymer computed characterizing parameters can be derived from the subgroup parameters, thus, also the subgroup computed characterizing parameters can refer to the same computed characterizing parameters as stated above. However, the computed characterizing parameters can also be derived without utilizing subgroups, for instance, by quantum chemical simulations of the whole polymer. In the following possible computed characterizing parameters are defined in more detail. Also in these cases the defined computed characterizing parameters can refer directly to the polymer computed characterizing parameters or, optionally, to the subgroup computed characterizing parameters.

A constitutional descriptor can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, torsion angle, branching degree, nucleotide and/or amino acid composition, nucleotide and/or amino acid sequence, nucleotide and/or amino acid sequence conservation, isoelectric point, glycosylation pattern, receptor binding constant,, Inhibitor constant,. A count descriptor can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers. Polymer descriptors referring to a list of structural fragment descriptors can refer to at least one of a list of molecular fractions, a list of functional groups, a list of bonds, and a list of atoms. Fingerprint descriptors comprise preferably, at least one of MACCS keys, preferably, in bit format or total amount format, Morgan and other circular fingerprints, preferably, in bit format or total amount format, topological torsion, atom pairs, infrared and related spectra, fingerprint count, PubChem fingerprint, substructure fingerprint, and Klekota-Roth fingerprint. Graph invariants/topological indices descriptors comprise preferably at least one of topostructural indices and topochemical indices. In a preferred embodiment the computed characterizing parameters comprise 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the polymer computed characterizing parameters refer to 3D descriptors comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or nonpolar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of polymer, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution.

In a further step, one or more test synthesis specifications associated with respective test polymers are determined from the plurality of potential test synthesis specifications based on the determined computed characterizing parameter values. For example, predetermined rules can be utilized that determine which of the potential test synthesis specifications are selected based on the computed characterizing parameter values. For example, the rules can indicate that, if two or more test synthesis specifications comprise one or more computed characterizing parameter values for one or more computed characterizing parameters lying within the same predetermined range, one of these potential test synthesis specifications is selected as a test synthesis specification. For such a case it is, for example, expected that due to the very similar computed characterizing parameter values also the measurements performed with respect to the test polymers will be very similar and thus it can be suitable to test or measure only one of these potential test polymers. Moreover, also preknowledge on functional relations between computed characterizing parameter values and, for instance, a target technical application property, can be utilized to select from the potential test synthesis specifications test synthesis specifications for which it can be expected that they might fulfil a respective target technical application property.

In a preferred embodiment, the one or more test synthesis specifications are determined based on the computed characterizing parameter values by performing a dimensional reduction with respect to the computed characterizing parameters and determining the one or more test synthesis specifications based on a resulting reduced parameter set comprising one or more computed characterizing parameters and/or combined quantities derivable from the computed characterizing parameters. A combined quantity is defined as a quantity that is derivable form the computed characterizing parameters by mathematically combining the computed characterizing parameters. For example, a linear combination of computed characterizing parameters results in a combined quantity. Generally, a dimensional reduction can be performed based on any known method for dimensional reduction, for example, a principle component analysis or a reduced rank linear discriminant analysis can be performed with respect to the computed characterizing parameters. Also a variable clustering analysis can be utilized for determining the reduced parameter set. The dimensional reduction then results in a reduced parameter set that comprises one or more computed characterizing parameters and/or combined quantities derived from the computed characterizing parameters. For example, in a dimensional reduction it can be determined that one of the main components for the potential test synthesis specifications refers to a combination of one or more computed characterizing parameters, in particular, to a linear combination. The resulting reduced parameter set allows, in particular, to determine test synthesis specifications that comprise, with respect to the reduced parameter set, similar characteristics, for example, can be regarded as belonging to the same class of test synthesis specifications with respect to the computed characterizing parameters. In such case, it can be expected that potential test synthesis specifications belonging to the same cluster found in the cluster analysis or comprising similar characteristics will also result in similar measurement results during the experiment series. Thus, from such classes or similar potential test synthesis specifications only a small number, for instance, only one potential test synthesis specification, can be selected as test synthesis specification to avoid performing experiments leading to the same measurement results. Preferably, the determining of the one or more test synthesis specifications based on the reduced parameter set comprises determining potential test synthesis specifications as test synthesis specifications that cover a space defined by the reduced parameter set according to a predetermined criterion. The predetermined criterion can refer to any criterion that allows to determine the covering of a space defined by the reduced parameter set. Preferably, the predetermined criterion refers to a diversity pick criterion that allows to select test synthesis specifications that are as different from each other as possible. However, also other predetermined criterions can be utilized. For example, the test synthesis specifications can be selected such that a predetermined average distance, like a Euclidian or Manhattan distance, between the respective test synthesis specifications in the space defined by the reduced parameter set is optimized. Moreover, for the selection of the test synthesis specifications also general linear models and general linear mixed models in connection with optimality criteria like A-optimality, D-optimality, G-optimality, I-optimality and V-optimality can be utilized. Additionally, also a Space-Filling-Design method can be used in order to have already measured data points within the space defined by the reduced parameter set to catch potential non-linearities. In particular, D-optimal designs can be augmented with space filling designs.

In a last step of the method, the one or more test synthesis specifications comprising instructions for controlling a synthesis of the respective one or more test polymers are then provided. For example, the one or more test synthesis specifications can be provided to a user, for example, via an output unit like a display. However, the one or more test synthesis specifications can also be provided to a storage for storing the one or more test synthesis specifications. Generally, synthesis specification are instructions for a laboratory operator to control and perform an experiment and as such go beyond mere presentation of information. Preferably, the providing of the one or more test synthesis specification comprises providing control data based on the one or more test synthesis specifications, wherein the control data is configured for controlling a synthesis system to perform a synthesis of the one or more test polymers based on the one or more test synthesis specifications. For example, the control data can be provided as part of the synthesis specification. An example, of such a synthesis specification is a synthesis specification provided as a json file.

Generally, the control signals, i.e. control data, can be provided in any format that allows to directly or indirectly control a synthesis system for synthesizing a test polymer in accordance with the test synthesis specification. For example, the control data can be provided in a format that allows a synthesis system management application to interpret the control signals and then to control the synthesis system, for example, a synthesis robot, accordingly to produce the test polymer. However, the control data can also be provided in a format that directly allows a control of the respective synthesis system to produce a test polymer, for instance, that directly allows to control components, for example, to start or stop a heating unit, open or close valves, start or stop a mixer, etc. Generally, the synthesis system can refer to any fully or partly automated synthesis system provided in a laboratory or industrial environment that is generally configured to produce polymers based on synthesis specifications. Further, it is preferred, that the control data is further configured for controlling an automatic measurement system for performing a test procedure for measuring the one or more technical application properties of the synthesised one or more test polymers. Providing the control data such that it further can control an automated measurement system to perform a test procedure for measuring the one or more technical application properties of the synthesized one or more test polymers allows for a complete automation of the experiment series while minimizing human intervention. Respective controllable and automatable synthesis and measurement systems are generally already available and can be utilized to perform the synthesis and the measurements. However, in some applications the controlling of the synthesis system and automatic measurement system can also refer to a machine guided human machine interaction process in which, for example, a human verifies synthesis steps or measurement steps performed by the synthesis system and the measurement system, respectively, or in which the control data initiates, for instance, via a notification, the user to perform one or more tasks in the synthesis process or measurement process that cannot be performed by the respective system itself, for example, moving a probe from one position to another, adding one or more substances, etc.

In an embodiment, the method further comprises receiving one or more measured technical application properties for the one or more test polymers and determining based on the one or more measured technical application properties a plurality of new potential test synthesis specifications and/or new computed characterizing parameters and repeating the determination of one or more test synthesis specifications based on the new potential test synthesis specifications and/or new computed characterizing parameters. Generally, a measured quantity is defined as a quantity that is directly measured or derived from direct measurements utilizing known functional relations or physical laws. For example, in a completely automated process, as described above, the control data can also be configured to initiate that the measurements of the one or more technical application properties of the automatic measurement system are again made available for further processing, for instance, by providing them to a storage, or making them available in a respective network, for instance, in a cloud environment. Generally, the determining of a plurality of new potential test synthesis specifications and/or new computed characterizing parameters based on the one or more measured technical application properties can be performed in accordance with predetermined rules that depend on the respective objective of the experiment series. In particular, if the respective objective of the experiment series refers to providing a training dataset for training a property determination model, the determining can be based on a result of the training of the property determination model based on the training data comprising the previously determined test polymers. For instance, an accuracy of the trained property determination model can be determined and if the accuracy does not meet a predetermined criterion, based on the deviation from the predetermined criterion a plurality of new potential test synthesis specifications and/or new computed characterizing parameters can be determined and the above described method can be repeated until the property determination model fulfils the respective predetermined criterion. In another preferred example, if the objective of the experiment series refers to determining a target polymer comprising a target value for a target technical application property, before determining new potential test synthesis specifications and/or new computed characterizing parameters the method can comprise comparing the measured technical application properties, i.e. the measured values of the technical application properties for the one or more test polymers, with the target value. If one of the test polymers meets the target value, it can be determined to abort the experiment series and to determine the respective test polymer as the target polymer. However, if none of the test polymers meets the target value, the method can comprise determining the test polymers which comprise a measured value for the technical application property coming next to fulfilling the target value based on a distance measure like Euclidean distances in the space of the computed characterizing parameter. For example, the next ten test polymers can be determined and new potential test synthesis specifications and/or new computed characterizing parameters can then be determined based on the next test polymers. In particular, starting from the next test polymers new potential test synthesis specifications can be generated by varying the process and ingredient parameters of the next test polymers within predetermined limits. In this way, the search for the test polymer can be successively narrowed down with each experiment series until the test polymer fulfilling the target value is found or an abortion criterion determines that in the respective search area it is unlikely to find a target polymer. In another example, the measured technical application properties can indicate that the starting potential test synthesis specifications are all too far, i.e. more than a predetermined limit, from the respective target technical application property. In this case the new potential test synthesis specifications can be determined such that they cover other overlapping or completely different areas of the space of test synthesis specifications than the previously used potential test synthesis specifications. For example, parameters of the previously used test synthesis specifications can be amended according to predetermined rules such that the newly generated potential test synthesis specifications strongly differ in at least one parameter from the previous potential test synthesis specifications. In this case is search space is not narrowed down but widened or displaced. Generally, during the search for one or more goals for the test synthesis specifications all the above changes in the search space can occur depending on the measurement results, for example, in a case the new potential test synthesis specifications can be determined such that the search space is displaced and then after a further measurement narrowed down. This optimization procedure allows, in particular, together with providing control data for automatically controlling the synthesis and measurement of the test polymers, a nearly complete automation of an experiment series with different objectives while minimizing the influence of human experts.

In an embodiment, the method further comprises receiving constraint information indicative of technical and/or productional constraints in the synthesis of a polymer and providing the plurality of potential test synthesis specifications associated with respective potential test polymers further based on the constraint information. For example, the potential test synthesis specification can be checked with respect to the constraint information whether or not the respective potential test synthesis specification fulfils the respective constraints and can then only be added to the plurality of potential test synthesis specifications if it fulfils the respective constraints. However, the respective constraint information can also already be taken into account when generating the potential test synthesis specifications, for example, by directly avoiding process and/or ingredient parameters that do not fulfil the technical and/or production constraints.

In a further aspect of the invention, an apparatus for providing one or more test synthesis specifications each associated with a test polymer for performing of an experiment series is presented, wherein a test synthesis specification is suitable for synthesising the respective test polymer and measuring one or more technical application properties of the test polymer in the experiment series, wherein the apparatus comprises i) an input interface configured for a) receiving synthesis cess parameters defining the ingredient and/or process parameter ranges in which one or more test polymers for the experiment series are to be determined, wherein different ingredient and/or process parameters are associated with different potential test polymers, b) deriving a plurality of potential test synthesis specifications associated with respective potential test polymers based on the synthesis parameters, c) providing computed characterizing parameters, wherein a computed characterizing parameter is indicative of a characteristic of a polymer and/or is derivable from one or more characteristics of the polymer, ii) one or more processors configured for a) determining for the provided computed characterizing parameters a computed characterizing parameter value for the potential test polymers based on the plurality of potential test synthesis specifications, b) determining one or more test synthesis specifications associated with respective test polymers from the plurality of potential test synthesis specifications based on the determined computed characterizing parameter values, and iii) an output interface configured for providing the one or more test synthesis specifications comprising instructions for controlling a synthesis of the respective one or more test polymers.

In a further aspect of the invention, a computer program product is presented for providing one or more test synthesis specifications, wherein the computer program product comprises program code means for causing an apparatus as described above to execute the method as described above.

In a further aspect of the invention, an interface method for providing one or more test synthesis specifications each associated with a test polymer for performing of an experiment series is presented, wherein the interface method comprises a) receiving, via an input unit, synthesis parameters defining the ingredient and/or process parameter ranges in which one or more test polymers for the experiment series are to be determined, b) interfacing, via an interface unit, with a processor performing the method as described above for providing the one or more test synthesis specifications comprising instructions for controlling a synthesis of the respective one or more test polymers, and c) providing, via an output unit, the one or more test synthesis specifications.

In a further aspect of the invention, an interface apparatus for providing one or more test synthesis specifications each associated with a test polymer for performing of an experiment series is presented, wherein the interface apparatus comprises a) an input unit configured to receive synthesis parameters defining the ingredient and/or process parameter ranges in which one or more test polymers for the experiment series are to be determined, b) an interface unit configured to interface with a processor performing the method as described above for providing the one or more test synthesis specifications comprising instructions for controlling a synthesis of the respective one or more test polymers, and c) an output unit configured for providing the one or more test synthesis specifications.

In a further aspect of the invention, a computer implemented method for generating a machine learning based determination model is presented, wherein the trained determination model is adapted to determine a technical application property of a polymer based on one or more polymer computed characterizing parameters, wherein a polymer computed characterizing parameter is indicative of a characteristic of a polymer and/or is derivable from one or more characteristics of the polymer, wherein the method comprises a) receiving model synthesis parameters defining the ingredient and/or process parameter range in which at least two training polymers for the training process are to be determined, wherein different ingredient and/or process parameters are associated with different potential training polymers, b) deriving a plurality of potential training synthesis specifications associated with respective potential training polymers based on the model synthesis parameters, c) providing computed characterizing parameters, d) determining for the provided computed characterizing parameters a computed characterizing parameter value for the potential training polymers based on the plurality of potential training synthesis specifications, e) determining at least two training synthesis specifications associated with respective training polymers from the plurality of potential training synthesis specifications based on the determined computed characterizing parameter values, f) receiving for the at least two training polymers measured technical application property values of the technical application property, g) training the machine learning based determination model by parameterizing the determination model based on computed characterizing parameters and the measured technical application property values of the at least two training polymers, and h) providing the trained determination model.

Generally, the determining of the at least two training synthesis specifications associated with respective training polymers comprises the same steps and can be performed in accordance with the same embodiments described above with respect to determine general test synthesis specifications. In particular, determining training polymers for training a machine learning determination model refers to a preferred application of the above described method for determining test synthesis specifications, wherein in this application the determined test synthesis specifications refer to training synthesis specifications. Generally, the determination model is a data driven model, wherein the term “data driven” is used to emphasize that the model is mainly based on respective data input and not, for instance, on intuition, personal experience, or knowledge. Preferably, the determination model is based on known machine learning algorithms, like neural networks, regression models, classification algorithms, etc. It has been found that for most applications in this context, in particular, regression models based on Linear Regression, Random Forests, Lasso, Boosted Trees, Ridge Regression and MARS algorithms are suitable, whereas for classification models, in particular, Random Forests, Logistic Regression, and SVM algorithms are suitable. Generally, the determination model is parameterized during the training process, wherein in the training process the determined training data set based on the measurements of the determined training polymers is utilized for the training of the determination model. Generally, any known and suitable training method can then be utilized to train the trainable property determination model based on the at least two training polymers, for instance, Newton algorithms, like steepest gradient methods, can be used.

Generally, in a preferred embodiment, further the trained determination model can be validated, for example, by applying the determination model to polymers that have not been part of the training polymers and for which respective measured technical application property values are known. If the trained determination model does not fulfil a predetermined criterion, for example, a predetermined accuracy, a plurality of new potential training synthesis specifications and/or of new characterizing parameters can be provided and the method of training the determination model can be repeated based on the plurality of new potential training synthesis specifications and/or new computed characterizing parameters. This iterative training method allows for an optimization of the training dataset utilized for training the determination model and thus also leads to a more accurate and reliable determination model.

In a further aspect of the invention, a training apparatus for generating a machine learning based determination model is presented, wherein the trained determination model is adapted to determine a technical application property of a polymer based on one or more polymer computed characterizing parameters, wherein a polymer computed characterizing parameter is indicative of a characteristic of a polymer and/or is derivable from one or more characteristics of the polymer, wherein the apparatus comprises i) an input interface unit configured for a) receiving model synthesis parameters defining the ingredient and/or process parameter range in which at least two training polymers for the training process are to be determined, wherein different ingredient and/or process parameters are associated with different potential training polymers, b) deriving a plurality of potential training synthesis specifications associated with respective potential training polymers based on the model synthesis parameters, c) providing computed characterizing parameters, d) determining for the provided computed characterizing parameters a computed characterizing parameter value for the potential training polymers based on the plurality of potential training synthesis specifications, ii) one or more processors configured to a) determining at least two training synthesis specifications associated with respective training polymers from the plurality of potential training synthesis specifications based on the determined computed characterizing parameter values, b) receiving for the at least two training polymers measured technical application property values of the technical application property, c) training the machine learning based determination model by parameterizing the determination model based on computed characterizing parameters and the measured technical application property values of the at least two training polymers, and iii) an output interface configured for providing the trained determination model.

In a further aspect, a computer program product for generating a machine learning based determination model is presented, wherein the computer program product comprises program code means for causing a computing system, in particular, an apparatus as described above, to execute a method as described above.

In a further aspect of the invention, an interface method for generating a machine learning based determination model is presented, wherein the interface method comprises a) receiving, via an input unit, synthesis parameters defining the ingredient and/or process parameter ranges in which one or more training polymers for the training of the determination model are to be determined, b) interfacing, via an interface unit, with a processor performing the method as described above for generating the determination model, and c) providing, via an output unit, the determination model.

In a further aspect of the invention, an interface apparatus for generating a machine learning based determination model is presented, wherein the interface apparatus comprises a) an input unit configured to receive synthesis parameters defining the ingredient and/or process parameter ranges in which one or more training polymers for the training of the determination model are to be determined, b) an interface unit configured to interface with a processor performing the method as described above for generating the determination model, and c) an output unit configured for providing the determination model.

In a further aspect of the invention, a computer-implemented method for controlling of an experiment series for determining a target polymer comprising a predetermined target technical application property is presented, wherein the method comprises a) receiving a target value for the target technical application property, b) receiving synthesis parameters defining the ingredient and/or process parameter ranges in which the target polymer is to be searched, wherein different ingredient and/or process parameters are associated with different potential target polymers, c) deriving a plurality of potential test synthesis specifications associated with respective potential test polymers based on the synthesis parameters, d) providing computed characterizing parameters, wherein a computed characterizing parameter is indicative of a characteristic of a polymer and/or is derivable from one or more characteristics of the polymer, e) determining for the provided computed characterizing parameters a computed characterizing parameter value for the potential test polymers based on the plurality of potential test synthesis specifications, f) determining one or more test synthesis specification associated with respective test polymers from the plurality of potential test synthesis specifications based on the determined computed characterizing parameter values, g) generating control data for controlling the experiment series based on the one or more test synthesis specifications, wherein the control data comprise instructions for controlling a synthesis of the respective one or more test polymers and the performing of the respective measurement of the target technical application property, h) receiving measured values for the target technical application properties for the one or more test polymers, i) comparing the measured values of the target technical application property of the one or more test polymers with the target value and, based on the comparison, either I) determining a test polymer of the one or more test polymers as the target polymer and the respective test synthesis specification as the target synthesis specification, or II) deriving a plurality of new potential test synthesis specifications and/or new physicochemical parameters and repeating the determination of one or more test polymers and the measuring of the target technical application property utilizing the new potential test synthesis specification of the new potential target polymer, and j) providing the determined target polymer and the target synthesis specification.

Generally, also the steps for determining the test synthesis specification corresponding to test polymers can refer to the same steps and the same embodiments described above for the general case of determining test synthesis specifications. In particular, the determining of a target polymer comprising a predetermined target technical application property only refers to a specific application of the experiment series and the determined test polymers utilizing a method as described above.

In particular, the comparison of the measured technical application property value with the target value allows to determine whether the measured technical application property fulfils a predetermined criterion, for example, that the measured technical application property meets the target value of the target technical application property within predetermined limits. If such a criterion is fulfilled, the respective test polymer is determined as the target polymer and the respective test synthesis specification as the target synthesis specification and the method proceeds to the next step. However, if the comparison indicates that the measured technical application property value does not meet the target value within the predetermined limits, a next iteration step utilizing new test synthesis specifications and/or new computed characterizing parameters has to be processed. In particular, for each iteration step of the iteration new test synthesis specifications are determined, preferably based on the previous test synthesis specifications, for instance, by amending one or more features of the previous test synthesis specification. For example, a predetermined number of test synthesis specifications can be determined for which the measured technical application property value is next to the target value, and the new synthesis specifications can be determined based on these test synthesis specifications, for example, by utilizing these as starting synthesis specifications. However, new potential test synthesis specifications can also be generated by arbitrarily choosing new potential test synthesis specifications from a huge amount of previously generated potential test synthesis specifications that lie within the process and ingredient parameter range. Moreover, also more sophisticated methods like Bayesian optimization can be utilized for selecting new potential test synthesis specifications for a further iteration step. Based on the new potential test synthesis specification and/or the new computed characterizing parameters, in each iteration step again test polymers are determined, a technical application property value is measured and the measured technical application property value is again compared with the target value such that the comparison can again lead to a further iteration step or if the respective criterion is fulfilled a respective new test polymer can be selected as the target polymer. Moreover, also an additional abortion criterion for the iteration can be selected, for instance, a number of iteration steps can be determined before the iteration is aborted with a notification to a user that no target polymer could be found for the respective technical application property. However, alternatively, after a predetermined amount of iteration steps the method can further comprise amending the target technical application property, for instance, by increasing the predetermined limits around the technical application property and to repeat the iteration while utilizing the increased limits during the comparison. Further, additional or alternatively to amending the target technical application property, the process and ingredient parameter ranges can be amended, for example, the ranges can be increased in order to allow for more potential test polymers to fall within the ranges. This can allow to find a target polymer that meets the technical application property as much as possible, even if a meeting of the original goal might not be possible. After the target polymer has been determined as described above, the target polymer and the target synthesis specification can be provided, for instance, to a user via an output unit.

Generally, the receiving of measurement values for technical application properties as performed by the methods above, i.e. the method for training a determination model and the method for determining a target polymer, can preferably refer to providing control data for initiating a controlling of a synthesis of one or more test or training polymers, respectively, based on the one or more test or training synthesis specifications, respectively, and further for initiating a testing process for measuring the technical application properties for the respective application for the one or more respective test or training polymers. In a preferred embodiment, the control data not only initializes but directly controls the synthesis process and the measurement process such that a complete automation of the generating of a determination model or of a determining of a target polymer can be achieved.

In a further aspect of the invention, a determination apparatus for controlling of an experiment series for determining a target polymer comprising a predetermined target technical application property is presented, wherein the apparatus comprises i) an input interface configured for a) receiving a target value for the target technical application property, b) receiving synthesis parameters defining the ingredient and/or process parameter ranges in which the target polymer is to be searched, wherein different ingredient and/or process parameters are associated with different potential target polymers, c) deriving a plurality of potential test synthesis specifications associated with respective potential test polymers based on the synthesis parameters, d) providing computed characterizing parameters, wherein a computed characterizing parameter is indicative of a computed characteristic of a polymer and/or is derivable from one or more computed characteristics of the polymer, ii) one or more processors configured for a) determining for the provided computed characterizing parameters a computed characterizing parameter value for the potential test polymers based on the plurality of potential test synthesis specifications, b) determining one or more test synthesis specification associated with respective test polymers from the plurality of potential test synthesis specifications based on the determined computed characterizing parameter values, c) generating control data for controlling the experiment series based on the one or more test synthesis specifications, wherein the control data comprise instructions for controlling a synthesis of the respective one or more test polymers and the performing of the respective measurement of the target technical application property, d) receiving measured values for the target technical application properties for the one or more test polymers, e) comparing the measured values of the target technical application property of the one or more test polymers with the target value and, based on the comparison, either I) determining a test polymer of the one or more test polymers as the target polymer and the respective test synthesis specification as the target synthesis specification, or II) deriving a plurality of new potential test synthesis specifications and/or new computed characterizing parameters and repeating the determination of one or more test polymers and the measuring of the target technical application property utilizing the new potential test synthesis specification of the new potential target polymer, and iii) an output interface configured for providing the determined target polymer and the target synthesis specification.

In a further aspect, a computer program product for determining a target polymer is presented, wherein the computer program product comprises program code means for causing a computing system, in particular, a determining apparatus as described above, to execute the method as described above.

In a further aspect of the invention, an interface method for controlling of an experiment series for determining a target polymer comprising a predetermined target technical application property, is presented, wherein the interface method comprises a) receiving, via an input unit, a target value for the target technical application property, and synthesis parameters defining the ingredient and/or process parameter ranges in which the target polymer is to be searched, b) interfacing, via an interface unit, with a processor performing the method as described above for providing the one or more target polymers, and c) providing, via an output unit, the target polymer and the target synthesis specification.

In a further aspect, control data for controlling an experiment generated utilizing any of the methods described above is presented. Preferably, the control data is structured to instruct a machine to perform a synthesis of a polymer based on a synthesis specification as part of the experiment.

In a further aspect, a test, training and/or target synthesis specification comprising instructions for controlling a synthesis of the respective one or more test polymers provided by any of the methods described above, respectively, is presented. Preferably, the synthesis specification comprises control data for controlling the synthesis.

In a further aspect, use of control data generated utilizing any of the methods described above for controlling a synthesis system, in particular, laboratory equipment, for producing the one or more test polymers in accordance with the one or more test synthesis specifications is presented.

It shall be understood that the methods as described above, the apparatuses as described above and the computer program products as described above have similar and/or identical preferred embodiments, in particular, as defined in the dependent claims.

It shall be understood that a preferred embodiment of the present invention can also be any combination of the dependent claims or above embodiments with respective independent claims.

These and other aspects of the present invention will be apparent from and elucidated with reference to the embodiments described hereinafter.

### BRIEF DESCRIPTION OF THE DRAWINGS

FIG. 1 shows schematically and exemplarily a flowchart of a method for providing one or more test synthesis specifications for performing an experiment series according to the invention,

FIG. 2 shows schematically and exemplarily a flowchart of a method for generating a machine learning based determination model according to the invention,

FIG. 3 shows schematically and exemplarily a flowchart of a method for determining a target polymer according to the invention,

FIG. 4 shows schematically and exemplarily a flowchart of a conventional design of experiment method,

FIG. 5 shows schematically and exemplarily differences of an exemplary method according to the invention with respect to the conventional design of experiment method,

FIG. 6 shows schematically and exemplarily a further embodiment of a method according to the invention,

FIG. 7 shows schematically and exemplarily a method for providing recipe information,

FIG. 8 to 13 show schematically and exemplarily possible repeating units for exemplary polymers, and

FIGS. 14 and 15 show schematically and exemplarily block diagrams of exemplary system architectures of systems utilizing the invention.

### DETAILED DESCRIPTION OF THE DRAWINGS

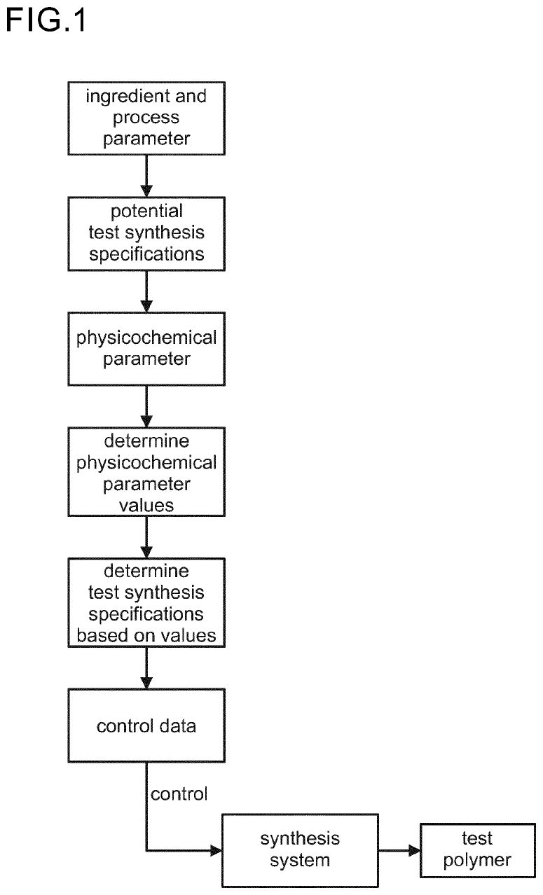

FIG. 1 shows schematically and exemplarily a flowchart of a computer implemented method for providing one or more test synthesis specifications each associated with a test polymer for performing of an experiment series. The method can be performed by any dedicated or general computing device, in particular, the method can also be performed not only by one processor of a computing device but by a plurality of processors, for example, in a distributed computing, like cloud computing or network computing.

In a first step, the method comprises receiving synthesis parameters defining ingredient and/or process parameter ranges in which one or more test polymers for the experiment series are to be determined. Generally, ingredient parameters can refer to parameters determining amount, food profile, and type of substances utilized for synthesizing the polymer, for example, to an amount, feed profile, and type of monomers, prepolymers, but also to catalysts and other additives. Process parameters refer to parameters indicative of the synthesis process of the polymer itself, for instance, to a temperature profile, a reactor type, a pressure profile, a stirring power, etc. utilized during the synthesis of the polymer. Preferably, the synthesis parameters are provided in accordance with an intended application of the results of the experiment series. However, it is further preferred that constraints, for example, technical or physical constraints, of the synthesis system that is utilized for the synthesis of a test polymer, are also already taken into account in the synthesis parameters. The defined ingredient and/or process parameter ranges thus indicate the search space in a process parameter and ingredient parameter space in which respective test synthesis specifications of test polymers are to be searched.

In a next step, a plurality of potential test synthesis specifications associated with respect potential test polymers are derived, in particular, generated, based on the synthesis parameters and thus based on the defined ingredient and/or process parameter ranges. In particular, from one or more starting points, i.e. starting potential test synthesis specifications, further potential test synthesis specifications can automatically be generated, for example, by varying ingredient and/or process parameters of the potential test synthesis specifications. The variation can be an arbitrary variation or can be based on predetermined rules like statistical design of experiment. Moreover, also a human expert can provide at least some of the potential test synthesis specifications, or can supervise the generating of the plurality of potential test synthesis specifications, for example, by providing new start potential test synthesis specifications for the generation, if necessary.

In a next step, computed characterizing parameters, preferably, associated with technical application properties, and thus with the objective of the experiment series, can be provided. For example, the computed characterizing parameters can be provided via an input unit by an expert based on preknowledge, known functional relations, or physical laws that relate the computed characterizing parameters to the technical application properties. A computed characterizing parameter can be regarded as being associated with the technical application property if an influence of the computed characterizing parameter on the respective technical application property is at least possible and cannot be ruled out, for example, based on known physical laws or previous experiments. In a next step, computed characterizing parameter values are determined for the provided computed characterizing parameters for the potential test polymers based on the plurality of potential test synthesis specifications. Generally, the determining of the computed characterizing parameter values can refer to accessing a storage on which computed characterizing parameter values for one or more potential test polymers are already stored and providing the respectively stored computed characterizing parameter values. However, it is preferred that the computed characterizing parameter values are determined, for instance, based on respective computations from the potential synthesis specifications. For example, for computed characterizing parameters referring to physicochemical parameters generally known methods for deriving physicochemical parameters from a target synthesis specification can be utilized, like molecular fingerprints or properties derived from the molecular connectivity, quantum mechanical simulations, molecular dynamics calculations, etc. However, if the computed characterizing parameters refer to subgroup parameters some computed characterizing parameters can also be directly derived from the potential test synthesis specification, for instance, in case of the computed characterizing parameters referring to an amount of a monomer in the test polymer or the amount of a certain chemical element or functional group.

In a next step, one or more test synthesis specifications are determined from the plurality of potential test synthesis specifications based on the determined computed characterizing parameter values. For example, predetermined or learned rules that depend on the respective objective of the experiment series can be utilized for determining the one or more test synthesis specifications based on the determined computed characterizing parameter values. In particular, statistical analysis methods can be utilized to statistically analyse the determined computed characterizing parameter values, for example, to determine correlations and/or similarity measures, wherein the test synthesis specifications can then be selected based on the statistical analysis, for example, based on the determined correlations and/or similarity measures. In particular, it is preferred that the test synthesis specifications are selected such that the computed characterizing parameter values associated with the selected test synthesis specifications are divers, i.e. do not comprise correlating or similar computed characterizing parameter values.

Preferably, before determining the test synthesis specifications based on the determined computed characterizing parameter values, a dimensional reduction with respect to the computed characterizing parameters is performed. In particular, a principle component analysis or similar known method can be utilized to identify a reduced parameter set that allows to cluster the potential test synthesis specifications with respect to the resulting reduced parameter set, for instance, by determining clusters along one or more of the members of the reduced parameter set. For example, if a plurality of clusters containing similarly behaving potential test synthesis specifications can be identified from the resulting reduced parameter set, the test synthesis specifications can be selected such that one test synthesis specification is selected from each cluster, whereas all other test synthesis specifications are ignored. Further, for example, also a variable clustering method can be utilized for determining the reduced parameter set and optionally, for determining clusters in the computed characterizing parameter values that can be used for selecting the test synthesis specifications. Preferably, the test synthesis specifications are selected based on the reduced parameter set such that they cover a space defined by the reduced parameter set according to a predetermined criterion, for example, a similarity criterion or distance criterion.

Generally, this determining of the test synthesis specification from the potential test synthesis specifications can be performed automatically but also in a user interaction process, for instance, a first selection of test synthesis specifications can be provided for a user for validation and the user can then amend the suggested test synthesis specifications, for instance, by adding or removing further test synthesis specifications or by choosing, for example, another criterion or statistical measure for determining the test synthesis specifications. The such determined test synthesis specifications can then be provided, for instance, as final result of the method to a user. However, it is preferred that further based on the test synthesis specifications control data are generated that allow for a direct or indirect control of a synthesis system to initiate the synthesis of the respective test polymers based on the respective test synthesis specifications.

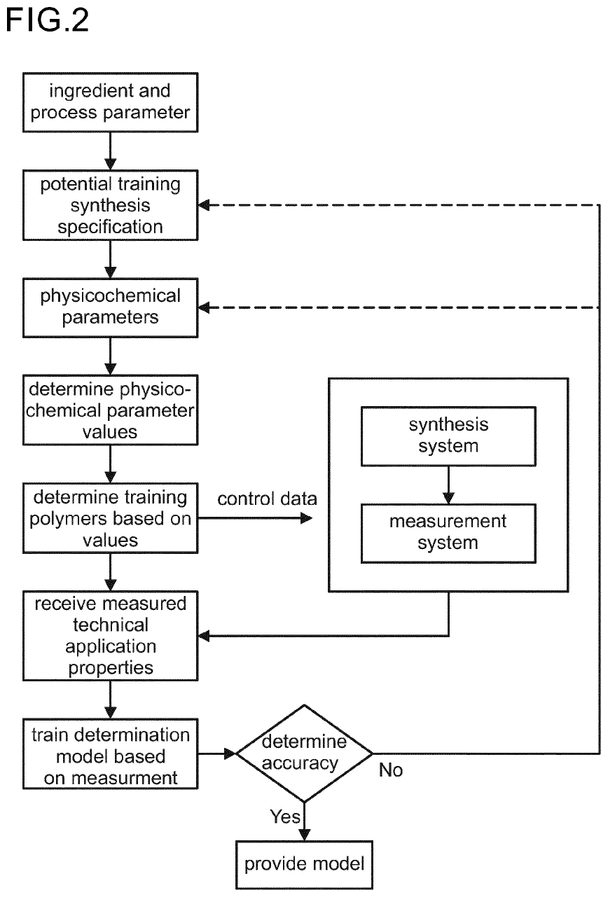

In a preferred embodiment, the above described method is applied for determining a training dataset for a machine learning based determination model and training such a machine learning based determination model based on the training dataset. FIG. 2 shows schematically and exemplarily a flowchart of such a preferred application. In particular, the steps of receiving synthesis parameters, providing potential training synthesis specifications, providing computed characterizing parameters, determining characterizing values for the computed characterizing parameters and determining training polymers based on the computed characterizing parameter values can be performed in the same way as described with respect to FIG. 1. In particular, the training polymers refer to the test polymers described above such that both methods are the same and the method for determining test polymers is only applied in the context of training a machine learning based determination model. In the method shown in FIG. 2 after the training polymers have been determined, in particular, in accordance with the method already described with respect to FIG. 1, control data can be provided to control or to initiate a controlling of respective laboratory equipment, in particular, of a synthesis system and a measurement system. Preferably, the control data are generated based on the determined training synthesis specifications of the respective training polymers in order to control the synthesis system such that the determined training polymers are synthesized. The synthesized training polymers can then be provided to a measurement system automatically or, for example, with the help of a respective user, and subjected to respective testing procedures for measuring the technical application property for which a machine learning based determination model should be trained. The respectively measured technical application properties are then provided from the measurement system again, for example, to an apparatus performing the method for generating a machine learning based determination model or to a dedicated apparatus performing the steps of training and evaluating the machine learning based determination model. The such received measured technical application properties are then utilized together with computed characterizing parameters of the respective test polymers in a training dataset for training the determination model. In particular, any known training method can be utilized, for instance, steepest decent methods or other respective Newton's algorithms can be utilized. After the determination model has been trained such that it can determine, based on one or more characterising parameter values the technical application property of a polymer, the respectively trained determination model can be provided and the method can end at this step.

However, it is preferred that in a further step the trained determination model is evaluated. In particular, a determination accuracy of the determination model can be determined. Generally, if the evaluation of the determination model is positive, for example, if the accuracy lies within predetermined limits (in FIG. 2 “Yes”), the determination model has been successfully trained and can be provided, for instance, to a respective storage to be later used in other applications. However, if the validation indicates that the trained determination model does not meet predetermined criterions (“No” in FIG. 2), the above described method can be repeated iteratively. In particular, new potential training synthesis specifications can be provided and/or new computed characterizing parameters can be utilized. The respective new potential training synthesis specifications and/or computed characterizing parameters can be determined in accordance with predetermined rules, for example, based on the previously utilized potential training synthesis specification and/or the previously utilized computed characterizing parameters. The determination of new potential training synthesis specifications includes an update of the selected computed characterizing parameters according to the selection and weighting of the computed characterizing parameters within the trained machine learning model. The new potential training synthesis are selected within the space of the updated set of computed characterizing parameters according to predetermined rules like diversity picks based on Euclidian distances in the space of the updated set of computed characterizing parameters. In particular, the trained determination model can provide information on the respective suitability of the used computed characterizing parameters. For example, the trained determination model can be trained with the same but also with different computed characterizing parameters than the characterizing parameters of the subset of computed characterizing parameters. The training of determination model can then utilize as a hyperparameter the search for the most relevant characterizing parameters for the determination of a respective technical application property resulting in a selection of respective computed characterizing parameters. In this case the selected characterizing parameters can be utilized in the next iteration step either for defining the new computed characterizing parameters or the utilized subset of the computed characterizing parameters. The method for determining the training dataset and then for training the determination model is then repeated based on the new potential training synthesis specifications and/or based on the new computed characterizing parameters. This iteration and optimization of the determination model can then be repeated until either the determination model passes the validation or another abortion criterion is fulfilled referring, for example, to an amount of repeating steps or an amount of determined test polymers. If no respective determination model can be provided at the end of the method, the method can also be repeated by amending the synthesis parameter, for example, by increasing the recipe and process parameter ranges for finding a respectively suitable training dataset.

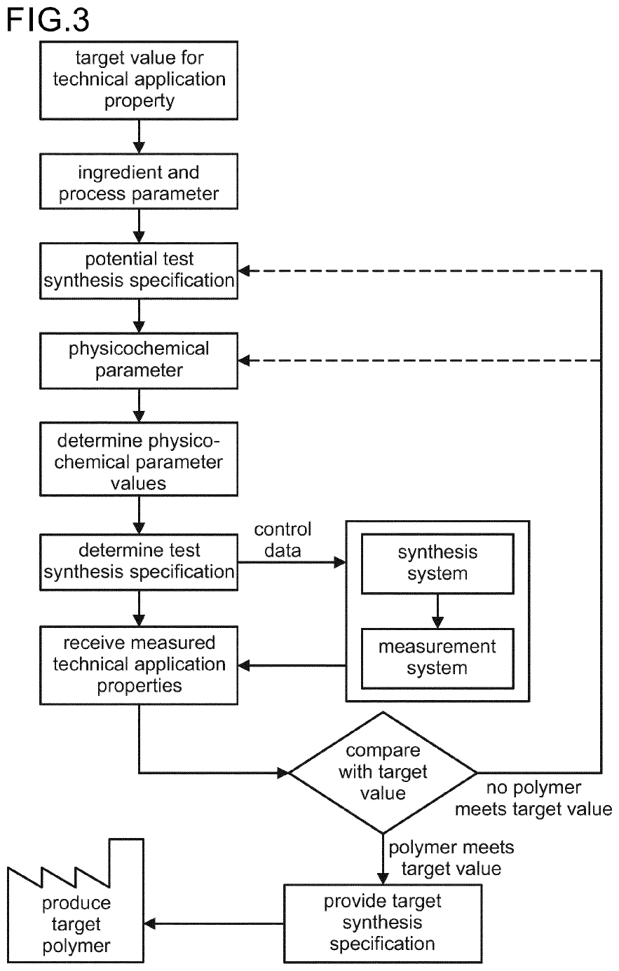

In another application of the method described with respect to FIG. 1, a target polymer is determined comprising a predetermined target technical application property, in particular, a predetermined target value of a target technical application property. A schematic and exemplary flowchart of this method is shown in FIG. 3. In a first step, the method comprises providing a target value for a target technical application property, for example, via a user interface. The following steps for the determining of the test synthesis specifications and thus of the test polymers are then also performed in accordance with the method and embodiments of the method described with respect to FIG. 1. After the test synthesis specification has been determined accordingly, control data is generated that is configured for controlling or for initiating a controlling, for example, a laboratory system comprising a synthesis system and a measurement system based on the determined test synthesis specifications. In particular, the synthesis system, for instance, a synthesis robot, is controlled to synthesize respective test polymers based on the test synthesis specifications and to provide the synthesized test polymers to the measurement system. The measurement system then applies the test polymers to one or more testing procedures to determine a measurement value for the test polymers for the target technical application property. The respective measurement values can then be again provided to an apparatus performing the method or to a dedicated apparatus for validating the measurement results of the test polymers. Based on the received measured technical application property values of the test polymers, it is determined whether or not one of the test polymers refers to the target polymer. In particular, for each of the test polymers the measured value is compared with the target value. If one or more of the measured values meet the target value the respective test polymers are determined as target polymers and the respective target synthesis specifications are provided. For example, the target synthesis specifications can be provided to a production system of a production plant in order to start a production of the target polymer on a bigger scale. However, if none of the test polymer comprises a measurement value meeting the target value within predetermined limits, a further optimization step can be initiated. In particular, new potential test synthesis specifications and/or new computed characterizing parameters can be provided. Preferably, the new potential test synthesis specifications and/or new computed characterizing parameters are provided based on the synthesis specifications and/or computed characterizing parameters of the previously selected test synthesis specifications and corresponding computed characterizing parameters. In particular, it can be determined which of the previously determined test synthesis specifications were closest to meeting the target value, e.g. by using a distance measure like Euclidean distances. For example, predetermined rules for defining such a closest test synthesis specification can be utilized, like determining the ten closest synthesis specifications or determining all synthesis specifications that lie within a predetermined limit around the target value. New potential test synthesis specifications and/or new computed characterizing parameters can then be determined based on these determined closest test synthesis specifications. For example, the determined closest test synthesis specifications can be utilized as starting test synthesis specifications for the generating of the new test synthesis specifications, wherein the variations are limited to a predetermined variation range. The method is then performed based on the provided new test synthesis specifications and/or computed characterizing parameters. This iteration can then be regarded as narrowing down the search space in which the target polymer is searched successively and efficiently until the target polymer can be determined.

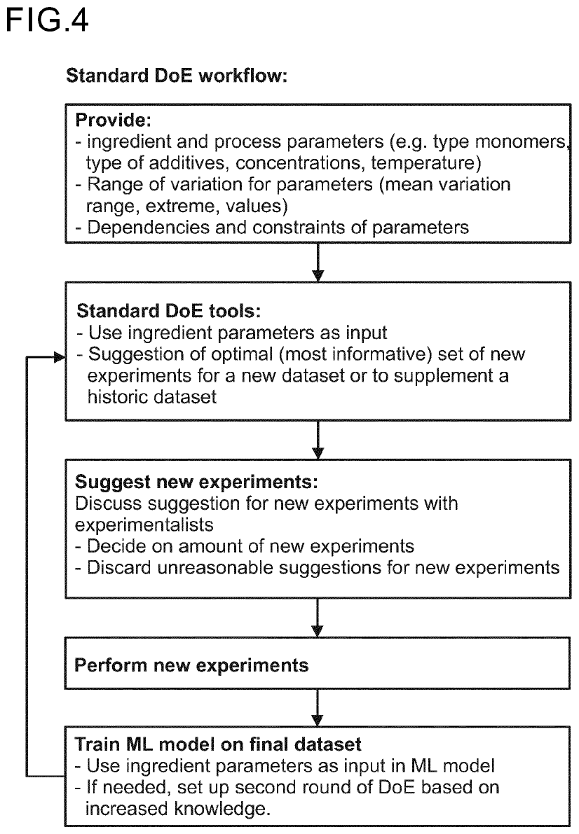

FIG. 4 shows schematically and exemplarily a flowchart of a commonly used design of experiment process. Such process is generally utilized for designing an experiment series for different objectives. Such a process is often based very strongly on the expertise of a respective expert for such processes. Generally, first synthesis parameters comprising ingredient and/or process parameters defining the ingredient and/or process parameter ranges are provided, for example, which type of monomers, type of additives, which amounts and which temperatures can be utilized for synthesizing the polymers, and a respective range of variation for the parameters is determined. Further, dependencies and constraints of these parameters can also be taken into account, for instance, technical constraints. In a next step, the synthesis parameters are utilized to suggest a set of new experiments depending, for example, on the respective objective of the design of experiment process. In particular, the suggestion of the new set of experiments is often based on the expertise, intuition and experience of a respective expert. Additionally, some standard statistical tools can be utilized to optimally cover the design space provided by the range of ingredient and process parameters. However, in this process the amount of necessary experiments scales with the amount of ingredient and/or process parameter ranges that can be varied for finding the respective target polymer, wherein with current methods it is often not possible to vary more than ten parameters to allow for a reasonable amount of experiments. In a next step of the commonly utilized design of experiment process the suggested new experiments can be discussed with experts for the experiments and it can be decided which of the suggested new experiments can be performed. During this process, some suggested experience might be discarded, for example, since experimental results are already known or since the experts indicate that a performance of the experiments might not be possible. The new experiments are then performed and utilized for the respective objective of the experiment series. In this example, the objective refers to the training of a machine learning determination model as in the example described with respect to FIG. 2. Thus, in this example the newly performed experiments are utilized in the training data for training the machine learning determination model. If a validation of the machine learning based determination model indicates that the training of the determination model was not successful with respect to one or more predetermined criterion, some steps of the design of experiment process can be repeated in an optimization routine. As already mentioned above, this common design of experiment process provides certain drawbacks, in particular, it is highly dependent on the experts performing the process and further still necessitates to perform a huge amount of experiments or to limit the number of possible variable process and ingredient parameters.

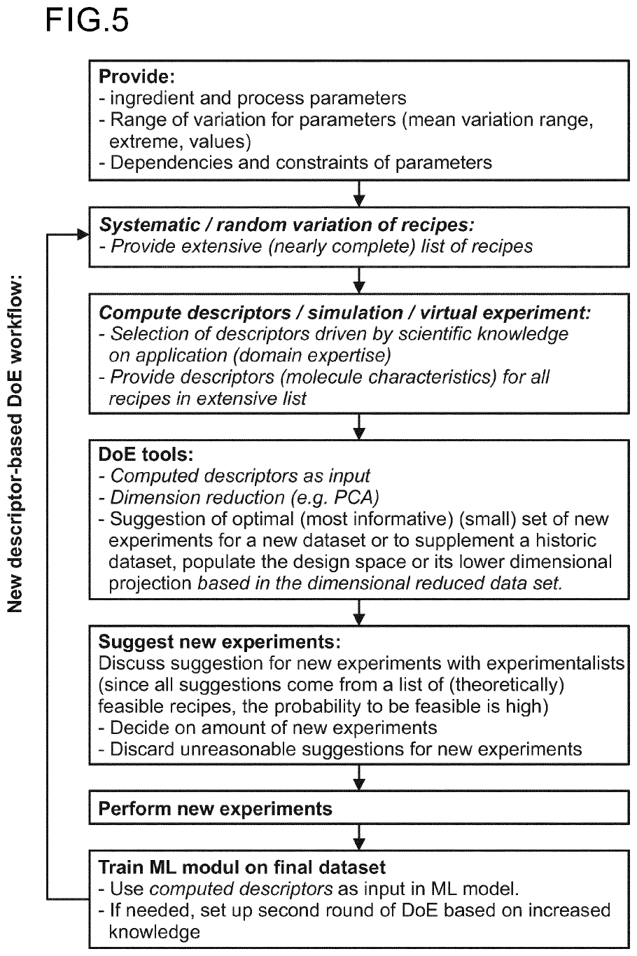

FIG. 5 shows schematically and exemplary the main differences between the commonly performed design of experiment process as described above and a design of experiment process performed in accordance with the new methods as described, for instance, with respect to FIGS. 1, 2 and 3. In particular, in a method according to the invention based on the synthesis parameters, an extensive list of possible experiments, i.e. synthesis specifications, is provided. Moreover, computed characterizing parameters, preferably, physicochemical descriptors, are provided, for example, based on expert or scientific knowledge with respect to a specific application and respective descriptor values are computed for the recipes in the list of recipes. Then, a dimension reduction can be performed with respect to the descriptors to find distinguishable clusters of the experiments or correlations and similarities between experiments in the list of experiments. Thus, based on the dimension reduced dataset, the new experiments can be suggested, for example, by avoiding to select experiments belonging to the same class, comprising strong similarities or correlations. Accordingly, a much smaller set of experiments can be suggested that still allows, from an objective point of view, to cover the space of possible experiments very well. The following steps are then again performed similar to a commonly known design of experiment process, in this example, in order to train a determination model. The difference introduced according to the invention into the design of experiment process not only allows to reduce the number of experiments that have to be performed extensively but also allows to select the to be performed experiments more objectively by minimizing the influence of an expert on the process. This further allows for a complete or semi-complete automation of the respective process.

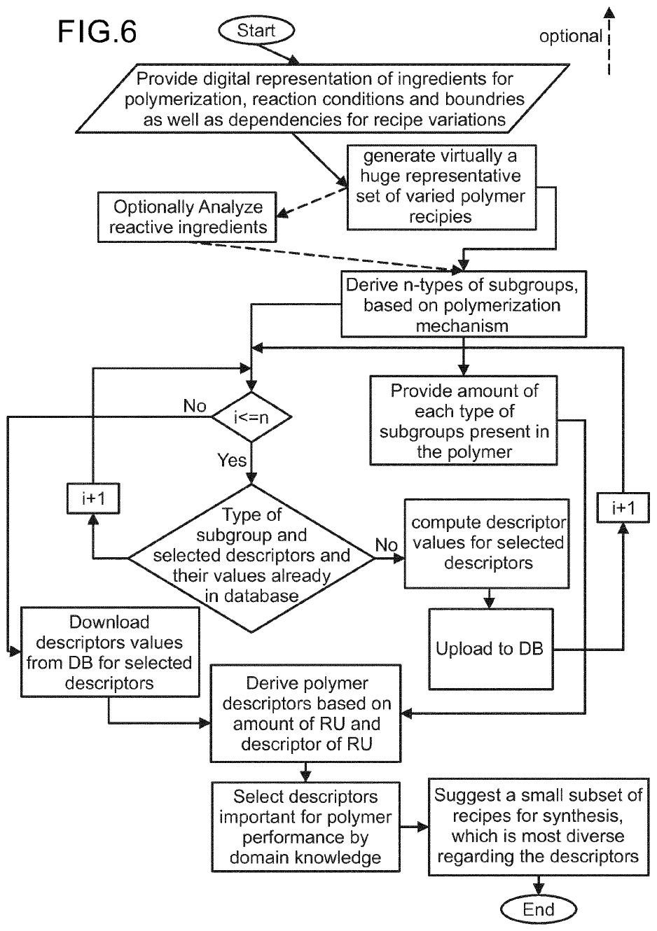

FIG. 6 shows schematically and exemplarily a flowchart of an embodiment of the invention referring to utilizing subgroups of a polymer for determining computed characterizing parameter values, referring in this case to physicochemical descriptors. In a first step 410 a digital representation of ingredients for polymerization, reaction conditions and boundaries is provided as well as dependencies for experiment variations. The digital representation is thus indicative for the process and ingredient parameters defining the process and ingredient parameter ranges, i.e. the search space. Based on this information, in a next step a huge representative set of varied polymer experiments, i.e. synthesis specifications, is virtually generated. In this context virtually is used to indicate that the generated synthesis specifications have not to be based on synthesized polymers. Based on the generated set of synthesis specifications one or more of the following information can be derived an amount of monomer components; an amount of non-monomer components, like initiators, fillers, additives; a reaction condition, like temperature, reactor type, pressure, stirring rate; condition profile, e.g. temperature profile, pH value, solvents; feed profiles; type of polymerization, e.g. radical, cationic, anionic, polycondensation, polyaddition, polyether formation; post-processing, like amount of components, conditions, as well as temperature and feed profiles; type of post-processing, e.g. radical, cationic, anionic, polycondensation, polyaddition, polyether formation; chemical information on components, like mixtures, connectivity of non-polymeric pure compounds, composition of polymeric pure compounds on the basis of subgroups, connectivity of the monomers associated to the subgroups in the polymeric pure components; for bock-co-polymers also information, in which block each monomer and reactive prepolymer is incorporated; for structures/layered materials and composites also information in which phase/layer each component is included. Moreover, in an optional step the reactive components can also be analyzed to determine information on the subgroups from the synthesis specification.

In a next step the polymerizable components can be transformed into subgroups, e.g. repeating units, and the subgroups are determined as different types. For example, polymerizable subgroups can be determined based on connectivity information of non-polymeric pure compounds by using SMARTS, for instance, via KNIME workflow. Also connectivity information of all possible subgroups can be derived from connectivity information of non-polymeric pure compounds by using reaction SMARTS, for example, also via KNIME workflow After the subgroups and their types have been determined, in a further step the computed characterizing parameters, e.g. physicochemical descriptors can be provided. However, the computed characterizing parameters can also be determined without first selecting the type of the subgroups. In order to decrease the computational resources for the method it is preferred that it is determined whether subgroup computed characterizing parameters associated with a respective type of subgroup are already stored in a database, for example, it can be determined if entries for subgroups with identical connectivity information already exist in the database. If this is the case the respective associated subgroup computed characterizing parameter can be directly downloaded. If the determined type of subgroup is not stored on the database the subgroup computed characterizing parameter that are associated with a respective type of subgroup can be determined. For example, either a 3D structure of respective types of subgroups can be derived based on connectivity information and an automatically computation of subgroup computed characterizing parameter can be started using, for instance, a computer cluster, or already existing machine-learning predictions can be utilized as subgroup computed characterizing parameter. Generally, it is preferred that if computations on new subgroups are necessary the results are stored in the database after the computations are finished. Optionally further subgroup computed characterizing parameters can be provided from a topological analysis of the subgroups, a quantum chemical computation, a molecular dynamics computation, coarse-grained methods, finite-element computations and kinetic simulations. In particular, polymer reaction engineering methods can be used to derive subgroup computed characterizing parameters that allow to take into account a microstructure of the polymer.

In a step the amount of subgroups, i.e. of each type of subgroup, is determined, for example based on the provided synthesis specification for the polymer. For example, the amount can be determined by counting an amount of polymerizable groups per polymerizable component, optionally, including prepolymers. In this case, information on polymerizable groups can be derived from non-polymeric components and the such determined amount can be added to a count of the number of, optionally, non-polymerized, polymerizable groups of the subgroups for polymeric components based on the composition of the polymeric components to determine a resulting amount. Further, it is preferred that the amount of polymerizable groups originating from agents used for post-processing after polymerization is removed from the resulting amount. The such determined amount of subgroups can be provided and, for example, saved on the database. Before further processing the determined amount of subgroups, subgroups which are completely represented by other subgroups can be removed. Moreover, subgroups, which have the same connectivity, can be merged.

Optionally the derived amounts of subgroups can be used for a further interpretation of the polymer composition. For example, a total number of polymerized functional groups, e.g. double bonds, amine groups, alcohols groups, thiol groups, carboxylic acid groups, isocyanate groups, epoxide groups, and formed functional groups, e.g. amid groups, ester groups, thioester groups, urea groups, urethane groups, thiourethane groups, ether groups, can be determined. Also the molar weighted total number of polymerized functional groups, the mass weighted total number of polymerized functional groups, the total number of residual functional groups, e.g. double bonds, amine groups, alcohol groups, thiol, groups, carboxylic acid groups, isocyanate groups, epoxide groups, the molar weighted total number of residual functional groups, the mass weighted total number of residual functional groups, the sum of all residual functional groups, the ratio between functional groups after polymerization, the number of crosslinks in polymer, the molar fraction of crosslinks in polymer, optionally, with mass-weighting as well, the average number of atoms per subgroup, optionally, per weight as well, the average number of non-H-atoms per subgroup, optionally, per weight as well, the average number of bonds per subgroup, optionally, per weight as well, the average number of bonds between non-H-atoms per subgroup, optionally, per weight as well, the average number of rotors per subgroup, optionally, per weight as well, the average number of rotors between non-H-atoms per subgroup, optionally, per weight as well, the average number of rings per subgroup, optionally, per weight as well, the average polar surface areas per subgroup, optionally, per weight as well, the average refractivity per subgroup, optionally, per weight as well, the total number of blocks, the molar size of first block, the molar size of last block, the HLB value of polymer, optionally, with area weighted HLB value, the HLB value of block with lowest HLB value, optionally, with area weighted HLB value, the HLB value of block with largest HLB value, optionally, with area weighted HLB value, the HLB value of first block, optionally, with area weighted HLB value, the HLB value of last block, optionally, with area weighted HLB value, the mass of first block, the mass of last block, the area of block with lowest HLB value, the area of block with largest HLB value, the difference of the HLB values of the blocks, optionally, with area weighted HLB value, the hydrophilic area of the polymer, the lipophilic area of the polymer, the number of arms for ring-opening-polymerization, or the length of arms for ring-opening-polymerization can be determined.

In a further step the determined amount and type of the subgroups and the associated subgroup computed characterizing parameters can be utilized to compute the polymer computed characterizing parameters. For example, the polymer computed characterizing parameters can be determined by one or more of molar weighted, e.g. arithmetic, harmonic or logarithmic, averaging, mass weighted, e.g. arithmetic, harmonic or logarithmic averaging, volume weighted, e.g. arithmetic, harmonic or logarithmic, averaging, surface area weighted, e.g. arithmetic, harmonic or logarithmic, averaging of the associated descriptors of the subgroups. Moreover, the polymer computed characterizing parameters can be determined by determining from the associated subgroup computed characterizing parameters one or more of a molar weighted standard deviation, a mass weighted standard deviation, a volume weighted standard deviation, a surface area weighted standard deviation, a molar weighted maximum value, a mass weighted maximum value, a volume weighted maximum value, a surface area weighted maximum value, a molar weighted minimum value, a mass weighted minimum value, a volume weighted minimum value, a surface area weighted minimum value, a molar weighted sum, a mass weighted sum, a volume weighted sum, a surface area weighted sum, and a maximal difference.

In a further step the derived or provided polymer computed characterizing parameters can then be utilized for selecting from the synthesis specification set, synthesis specification for an experimental series. In particular, computed characterizing parameters important for polymer performance can be selected, for example, based on domain knowledge. The domain knowledge can be gained, for instance, by statistical methods like dimension reductions with respect to the computed characterizing parameters. Thus, a reduced set of computed characterizing parameters is determined. Based on the reduced set of computed characterizing parameters, a small subset of recipes for synthesis, which is most diverse regarding the computed characterizing parameters, can be determined. In particular, statistical measures can be utilized to determined and optimize the diversity of the synthesis specifications with respect to the reduced set of computed characterizing parameters. The such selected and suggested synthesis specifications can then be provided for synthesis and can be utilized in the respective experiment series.

Preferably the polymer computed characterizing parameters utilized in the above described method originate from quantum chemical computations with solvation treatment. Quantum chemical computations scale very unfavorable with the system size, which makes computations on polymers or shorter monomer sequences impractical. This obstacle is solved by the above method that comprises cutting the polymer at preferably non-polarized bonds into subgroups. The resulting subgroups have a similar size than the monomers and the computed characterizing parameters can be calculated using quantum chemical methods.

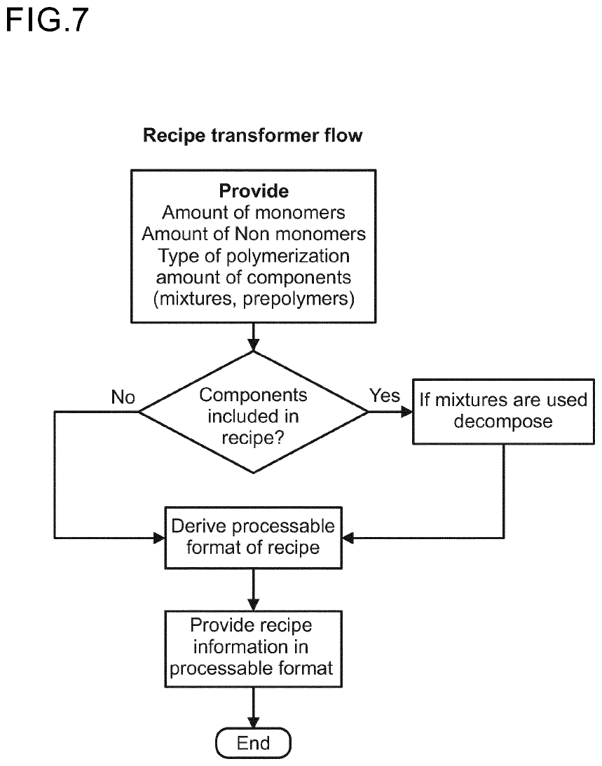

FIG. 7 shows schematically and exemplarily a method for providing recipe information, and determining a respective usable format for the recipe information. In a first step information indicative of recipe and/or process parameters is provided. In particular, it is preferred that the provided information comprises at least one of information on an amount of monomers, an amount of non-monomers, a type of polymerization, and an amount of components, e.g. mixtures, prepolymers, Based on this information it is determined whether the components of the recipe are provided in the information. If this is the case it is further determined if the information indicates a mixture. If this is the case the mixture is decomposed, i.e. the components of the mixture are determined, for example, based on predetermined knowledge and respective rules. After the decomposition of the mixtures or if the components are not directly provided by the information, a processable format for the recipe is determined. In particular, the format used for determining the physicochemical parameters can be derived. The respective recipe format can then be used in the following procedure, for example, as described above. This method step can be utilized, for example, as an interface method if recipe data from different not standardised sources is utilized. For example, if a customer provides the recipe data, it might be necessary to first derive a respective format for the information such that the above described methods can be performed based on the information provided by the customer. In an example, a customer might be interested in some goal, wherein a fixed mixture of “C10-C12 carboxylic acid” in a specific mass interval should be the utilized and provide the respective recipe information. Generally, “C10-C12 carboxylic acid” comprises 30% C10 carboxylic acid, 40% C11 carboxylic acid and 30% C12 carboxylic acid. If this information is not provided by the information provided by the customer, it can be derived, for example when deriving the components of the mixture. In another example, instead of a classic mixture, the information can indicate the usage of partially protonated substances, e.g. amine, as mixture of a protonated and a non-protonated substance. Also in this case the respective parts of the mixture as components are derived. Since in most cases it is easier to calculate the physicochemical parameters for pure substances instead of mixtures, after determining the components of the recipes provided by a customer, a respective format of the recipe can be derived and provided that allows to further process the recipe information. In the example above, the information referring to “C10-C12 carboxylic acid” can, for instance, be replaced by the partial amounts of the three pure substance forming this mixture. The respective physicochemical parameters can then be determined based on the new formatted recipe, in particular, based on the pure substance. Generally, some recipe information can also be provided by providing information on prepolymers that are utilized during the polymerization. Also the components of these prepolymers, for example, the respective repeating units but also the average functionality of the prepolymers, can then be determined and the recipe provided in a format also providing this information. This allows for a faster determination of the physicochemical parameters.

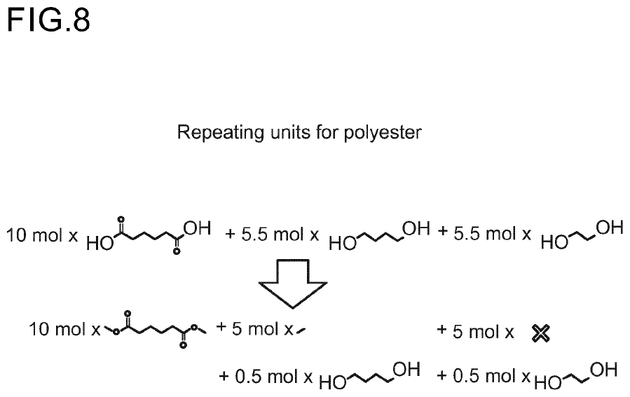

In the following some examples of subgroups determined for specific polymers are described in more detail with respect to FIG. 8 to 13. In particular, these below provided examples on the deriving of subgroups are only exemplarily and utilize models and assumptions that lead to suitably accurate prediction results. However, also other models and assumptions can be used for deriving subgroups. In particular, utilizing kinetic models for deriving the subgroups allows for a further increase of an accuracy when determining the subgroups and also can increase the accuracy of the prediction result. FIG. 8 shows the polymerized monomers derived, for example, from a provided number of monomers for a polycondensation. In this example 10 mol adipic acid is polymerized with 5.5 mol butane-1,4-diol and 5.5 mol ethylene glycol. The 10 mol adipic acid monomers polymerize to form 10 mol adipic acid dimethyl ester, i.e. the polymerized monomers of adipic acid in the resulting polymer. The additional carbon atoms of the polymerized monomer are taken from the monomers containing alcohol groups. Accordingly, the polymerized monomer of the monomer butane-1,4-diol within the polymer chain is ethane. In the case of ethylene glycol, the polymerized monomer within the polymer chain is completely represented by the polymerized adipic acid monomer (=adipic acid dimethyl ester), which is symbolized by the cross in the scheme. This polymerized monomer of ethylene glycol can be neglected for the computation of descriptors. According to the provided number of monomers, there is an excess of 2 mol of alcohol groups compared to the acid groups. Assuming a hypothetic complete polymerization of adipic acid and an equal reactivity of the two diols butane-1,4-diol and ethylene glycol, 0.5 mol of butane diol and 0.5 mol of ethylene glycol remain unreacted. These unreacted monomers resemble polymerized monomers of the polymer chain ends. Besides this assumption it is also possible to get a more realistic distribution of polymerized monomers, for instance, with kinetic models.

FIG. 9 shows the polymerized monomers derived, for example, from a provided number of monomers for a polyaddition. In this example 10 mol hexamethylene diisocyanate is polymerized with 6 mol cyclohexane-1,4-diol, 3 mol glycerol and 2 mol butane1,4-diamine. In this example, it is assumed that the amines react preferably with isocyanates than alcohols. In a first step, 2 mol hexamethylene diisocyanate polymerize to a urea group containing polymerized monomer. Within this polymerized monomer the formed urea groups are N-substituted by methyl groups, which are taken from the amine containing compounds. Consequently, the monomer butane1,4-diamine polymerizes to ethane as polymerized monomer, since the original amine groups and two of the four carbon atoms of the monomer 1,4-butanediamine are attributed to the polymerized monomer of hexamethylene diisocyanate already. In a second step, the alcohol groups polymerize with the remaining 8 mol hexamethylene diisocyanate. There is an excess of alcohol groups compared to the number of isocyanate groups. Therefore, the 8 mol hexamethylene diisocyanate polymerize to 8 mol urethane group containing polymerized monomers which are O-substituted by methyl groups. Under the assumption of a hypothetical equal reactivity of cyclohexane-1,4-diol and glycerol, 4.57 mol polymerized cyclohexane-1,4-diol is formed, which is represented by cyclohexane. In this special case, no carbon atom is removed from the monomer, because such a removal would change the size of the ring of cyclohexane-1,4-diol. We assume a similar reactivity of all three alcohol groups of glycerol, which results in a hypothetic reaction of all three alcohol groups with isocyanates. For each reacted alcohol group, a methoxy group is removed from glycerol. Consequently, the 2.29 mol polymerized glycerol is completely represented by the urethane containing polymerized monomer of hexamethylene diisocyanate and can be neglected for the computation of descriptors. Because of the excess of alcohol groups, 1.43 mol cyclohexane-1,4-diol and 0.71 mol glycerol remain unreacted. These unreacted monomers resemble polymerized monomers of the polymer chain ends. Besides these assumptions it is possible to get a more realistic distribution of polymerized monomers with kinetic models. In this example, 2.29 mol of polymerized glycerol is formed as described above. This polymerized monomer has three reacted functional groups and acts as cross-link in the final polymer. This information on cross-links can be used as descriptor to distinguish between linear and crosslinked polymers.

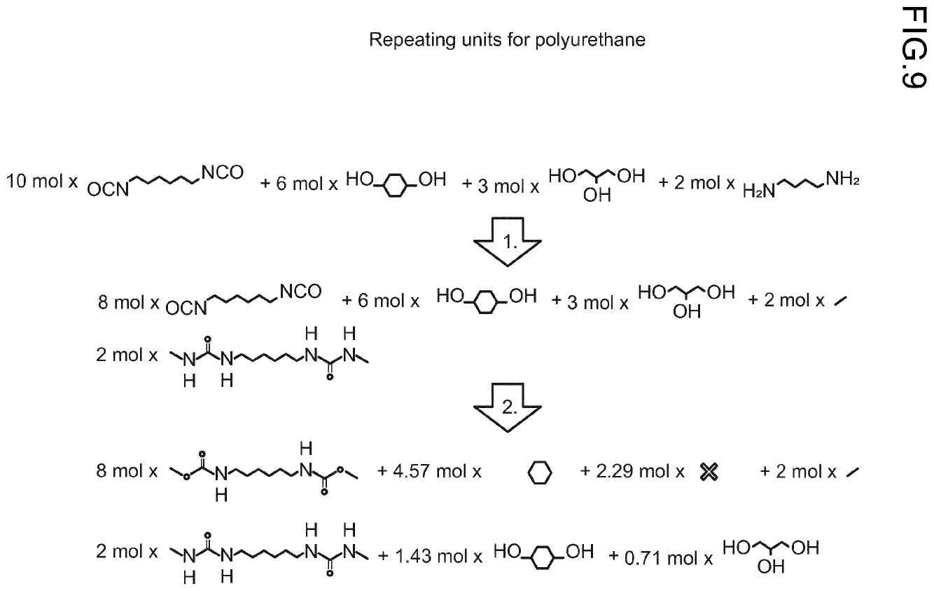

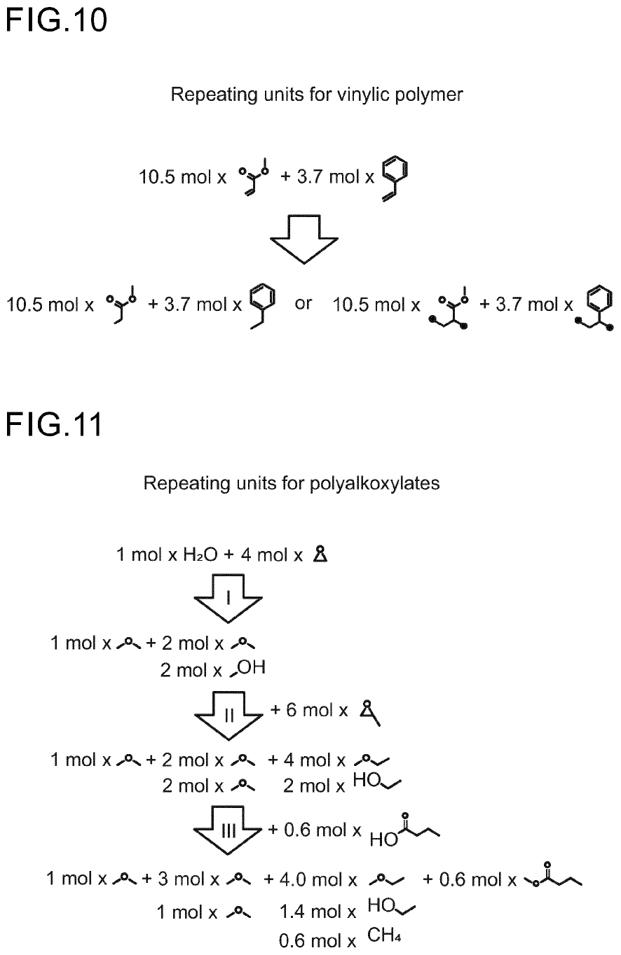

FIG. 10 shows the polymerized monomers derived from a provided number of monomers for a vinylic polymerization. In this example 10.5 mol methyl acrylate is polymerized with 3.7 mol styrene. In this example, a complete conversion of the monomers is assumed during polymerization. Therefore, 10.5 polymerized methyl acrylate and 3.7 polymerized styrene is formed. Polymerized monomers can be defined in different ways. On the left-hand side, the polymerized monomers are represented by molecular structures in which the reactive double bond of the corresponding monomers has been saturated upon a hypothetical hydrogenation (addition of 2 hydrogen atoms). For example, the monomer styrene can be represented by ethylbenzene as polymerized monomer. On the right-hand side, additional groups, e.g., methyl groups, are added, which resemble the electronic effect of the polymer chain on the polymerized monomer. However, these additional groups are preferably neglected by the descriptor computation, e.g., by ignoring their contribution to the molecular surface area. Besides this assumption it is also possible to get a more realistic distribution of polymerized monomers with kinetic models.

FIG. 11 shows the polymerized monomers derived from a provided number of monomers for a block-wise polyalkoxylation. In the first step, water is used as a model initiator representing e.g., hydroxy salts, of a polyalkoxylationin which 4 mol of ethylene oxide are polymerized. The polymerized monomer of water is dimethyl ether. After polymerization of the 4 mol ethylene oxide, two of them are located within the polymer chain and two are at the chain ends. The polymerized monomer of ethylene oxide within the chain is dimethyl ether as well. The polymerized monomer of ethylene oxide at the chain end is methanol. All polymerized monomers are attributed to the inner block of the final block-co-polymer. In a second step, 6 mol of propylene oxide reacts with the polymer from step 1. Now, the polymerized monomers at the chain end from step 1 react with propylene oxide. Consequently, these polymerized monomers at the chain end of step 1 are now located within the polymer chain and the resulting polymerized monomer is again dimethyl ether. Regarding the 6 mol of propylene oxide, 4 mol of them form polymerized monomers within the polymer chain (methoxyethane) and 2 mol form polymerized monomers at the chain end (ethanol). All polymerized monomers deriving from the monomer propylene oxide are attributed to the outer blocks of the block-co-polymer resulting after step 2. In a third step, a chain-end modification is performed of the block-co-polymer formed in the first two steps. This chain-end modification is done via a partial esterification with 0.6 mol of butyric acid, a condensation reaction under the loss of one water molecule per newly formed ester bond. The polymerized monomer of butyric acid after esterification is methyl butyrate. The additional oxygen and carbon atoms of this polymerized monomer are taken from the polymerized monomers containing alcohol groups, which are the polymerized monomers of propylene oxide at the chain end of the polymer resulting after step 2, i.e. ethanol. Accordingly, those of the polymerized monomers (ethanol) which have formed esters upon partial esterification are transformed to methane now. Besides this assumption it is possible to get a more realistic distribution of polymerized monomers also with kinetic models.

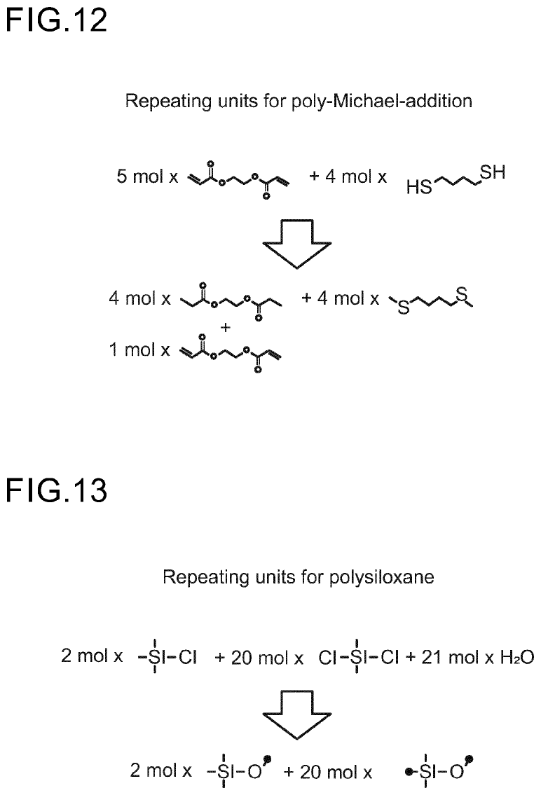

FIG. 12 shows the polymerized monomers derived from a provided number of monomers for a poly-Michael addition. In this example 5 mol ethylene glycol diacrylate is polymerized with 4 mol butane-1,4-dithiol. There is an excess of Michael-acceptor groups (acrylate groups) over Michael-donor groups (thiol groups). In this example, it is assumed that the 4 mol butane-1,4-dithiol are completely converted during polymerization. The resulting polymerized monomer of the monomer butane- 1,4-dithiol is 1,4-bis(methylsulfanyl)butane.

The additional carbon atoms of this polymerized monomer are taken from the monomers containing Michael-acceptor groups. Consequently, the reacted 4 mol of the monomer ethylene glycol diacrylate form 4 mol of ethylene glycol diacetate as polymerized monomers (loss of carbon atoms). The remaining excess of 1 mol ethylene glycol diacrylate monomers does not react. These unreacted monomers resemble polymerized monomers of the polymer chain ends. Besides this assumption it is also possible to get a more realistic distribution of polymerized monomers with kinetic models.

FIG. 13 shows the polymerized monomers for polysiloxanes. In this example 20 mol dichlorodimethylsilane polymerizes with 2 mol chlorotrimethylsilane and 21 mol water (hydrolysis and subsequent polyaddition). In this example, a complete conversion of the monomers is assumed during polymerization. Therefore, in this example, 20 polymerized monomers dichlorodimethylsilane within the polymer chain as well as 2 mol polymerized monomers chlorotrimethylsilane at the chain ends (represented by hydroxytrimethylsilane) are formed. Besides this assumption it is possible to get a more realistic distribution of polymerized monomers with kinetic models. In this example, it is not possible to define the polymerized monomers within the polymer chain in a way that the polymer is cut at non-polarized and homogeneous chemical bonds. Therefore, additional groups (e.g., methyl and methoxy groups) are added, which resemble the electronic effect of the polymer chain on the polymerized monomer. However, these additional groups must be neglected by the descriptor computation (e.g., by ignoring their contribution to the molecular surface area). By using methyl and methoxy groups as a model for the polymer chain, the polymerized monomer of dichlorodimethylsilane is dimethoxydimethylsilane. Alternatively, small oligomers can be used as polymerized monomer of dichlorodimethylsilane and chlorotrimethylsilane.

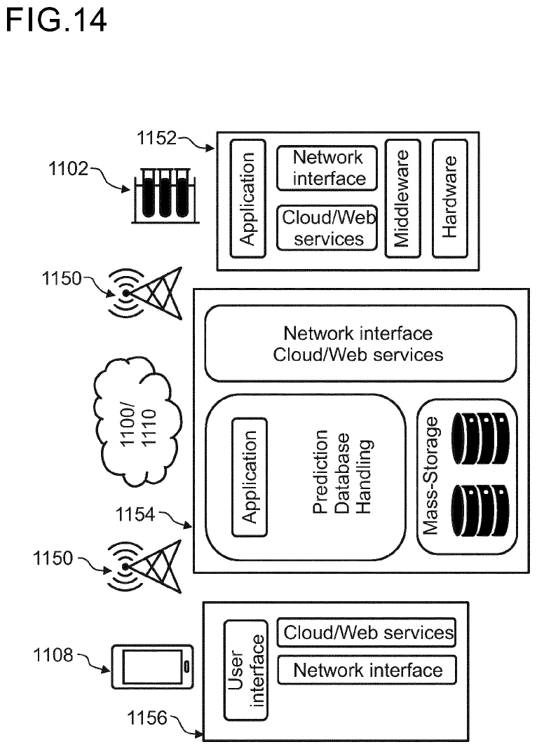

FIG. 14 illustrates a block diagram of an exemplarily system architecture of an automated laboratory system 1000 for synthesizing a polymer with a laboratory equipment control device 1102, a network 1150 and the synthesis specification, i.e. experiment, module 1100/1110, and a client device 1108. The automated laboratory system includes a laboratory equipment control device layer 1152 as part of the laboratory equipment control device 1102 as well as a synthesis specification module layer 1154 associated with the synthesis specification module and a remote control or client layer 1156 associated with the client device 1108. The laboratory equipment control device layer can be split into several hierarchical layers: the hardware, the middleware and the interface layer. The hardware layer relates to hardware resources such as sensors and actuators, in particular for controlling a synthesis of a polymer. The middleware relates to any of the known middleware for laboratory or plant synthesis operations. One example is LABS/QM, providing different abstractions to hardware, network and operating system such as low-level device control and message passing. The communication layer relates to communication protocols, wherein the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The synthesis specification module layer 1154 may include: a mass storage layer, the computing layer, the interface layer. The storage layer is configured to provide mass storage for the plurality of synthesis specifications from which the to be provided synthesis specifications are selected, as described in detail above. In particular, the functions performed by the apparatus, as described above, can be provided as program code means stored on the mass storage. Furthermore, synthesis specifications for a plurality of polymers can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on objectives for an experiment series. Such functionalities can include determining test polymers from a plurality of potential test polymers based on computed characterizing parameters of the potential test polymers, providing test synthesis specifications for the test polymers, and providing the test synthesis specifications as control data, i.e. control signal, to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the synthesis specification module layer 1154 or the laboratory equipment control device layer 1152. Users may be provided with a UI for selecting an experiment series objective, for example, the determining of a target polymer comprising a specific target technical application property value, and further for selecting process and/or ingredient parameters for defining a search space for the experimental series. In other examples, the users may be provided with a UI for selecting more than one objective and respective values. The applications may be configured for users to monitor and control the laboratory equipment control device and the operation remotely. In other examples, the client device layer and the synthesis specification module layer may be integrated into one device. The alternatives described here are only for illustration purposes and should not be considered limiting.

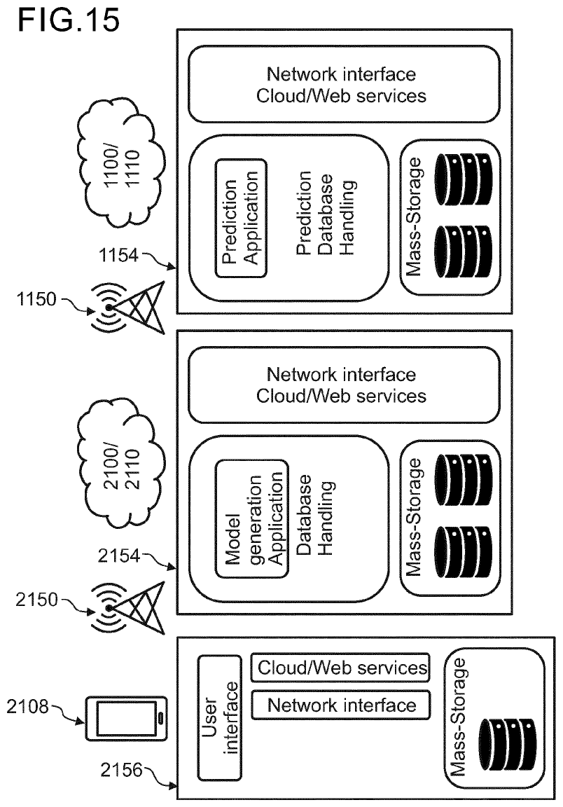

FIG. 15 illustrates a block diagram of an exemplarily system architecture of a system and apparatus for generating a determination model for determining a technical application property, a network 2150 and a model generating module 2100/2110 that can be regarded as or comprising a training apparatus, a synthesis specification module 1100/1110, and a client device 2108. The system for generating a determination model includes a model generating module layer 2154 as part of model generating module and a client layer 2156 associated with the client devices 2108.

The model generating module layer 2154 may include: a mass storage layer, a computing layer, an interface layer. The storage layer is configured to provide mass storage for the data-driven determination model as described above. Furthermore, the mass storage is configured for storing synthesis specifications for polymers and technical application properties. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a determination model for determining properties of polymers. Such functionalities may include receiving for at least two previously selected, in particular, in accordance with the above described method, and measured training polymers the measurement data of at least one technical application property for each of the at least two previously measured test polymers, training the model according to the above described training principles based on the at least two previously measured test polymers and the at least one technical application property for each of the at least two previously measured test polymers, and providing via an output interface the determination model for the technical application property. The model generating module layer may be configured for deploying the generated model and the synthesis specification database to the synthesis specification module layer. This may include storing the generated model and the synthesis specification database in the mass storage devices associated with the synthesis specification module.

The model generating module layer may further be configured for determining a digital representation of the polymer associated with the synthesis specification from the synthesis specification. The digital representation may include a set of polymer computed characterizing parameters and polymer computed characterizing parameter values associated with a synthesis specification of each measured polymer. One way of deriving these polymer computed characterizing parameters can be to apply the SMILES algorithm or any other already above described principle. In case, where the model is generated based on the synthesis specification, a relation between the synthesis specification and the computed characterizing parameters may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain synthesis specifications for polymers, and for at least two polymers at least one technical application property. The client layer further provides an interface for end-users. For end-users, the client layer 2156 may run client side Web applications, which provide interfaces to the model generation module layer 2154 or the mass storage devices associated with the client layer. Users may be provided with a UI for selecting a technical application property. The user may further be provided with a UI for selection of the synthesis specification data and the technical application property data associated with the synthesis specification data. The user interface may also provide an option for uploading the selected data to the model generating module layer and optionally an option to initiate model generation.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of process and ingredient parameters, the generating of the potential test synthesis specifications, the providing of the computed characterizing parameters, the determining of the computed characterizing parameter value, the determining of the test synthesis specifications, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to a method for providing test synthesis specifications for performing an experiment series. Synthesis parameters defining the ingredient and/or process parameter ranges in which test polymers for the experiment series are to be determined are provided. A plurality of potential test synthesis specifications are provided based on the synthesis parameters. Computed characterizing parameters are provided indicative of a computed characteristic of a polymer. A computed characterizing parameter value is determined for the provided computed characterizing parameters based on the plurality of potential test synthesis specifications. Test synthesis specifications are selected from the plurality of potential test synthesis specifications based on the determined computed characterizing parameter values and the test synthesis specifications are provided.

1. A computer implemented method for providing one or more test synthesis specifications each associated with a test polymer for performing of an experiment series, wherein a test synthesis specification is suitable for synthesising the respective test polymer and measuring one or more technical application properties of the test polymer in the experiment series, wherein the method comprises:
receiving synthesis parameters defining ingredient and/or process parameter ranges in which one or more test polymers for the experiment series are to be determined, wherein different ingredient and/or process parameters are associated with different potential test polymers,
deriving a plurality of potential test synthesis specifications associated with respective potential test polymers based on the synthesis parameters,
providing computed characterizing parameters, wherein a computed characterizing parameter is indicative of a computed characteristic of a polymer and/or is derivable from one or more computed characteristics of the polymer
determining for the provided computed characterizing parameters a computed characterizing parameter value for the potential test polymers based on the plurality of potential test synthesis specifications,
determining one or more test synthesis specifications associated with respective test polymers from the plurality of potential test synthesis specifications based on the determined computed characterizing parameter values,
providing the one or more test synthesis specifications comprising instructions for controlling a synthesis of the respective one or more test polymers.

2. The method according to claim 1, wherein the one or more test synthesis specifications are determined based on the computed characterizing parameter values by performing a dimensional reduction with respect to the computed characterizing parameters and determining the one or more test synthesis specification based on a resulting reduced parameter set comprising one or more computed characterizing parameters and/or combined quantities derivable from the computed characterizing parameters.

3. The method according to claim 2, wherein the determining of the one or more test synthesis specifications based on the reduced parameter set comprises determining potential test synthesis specifications as test synthesis specifications that cover a space defined by the reduced parameter set according to a predetermined criterion.

4. The method according to claim 1, wherein the providing of the one or more test synthesis specification comprises providing control data based on the one or more test synthesis specifications, wherein the control data is configured for controlling a synthesis system to perform a synthesis of the one or more test polymers based on the one or more test synthesis specifications.

5. The method according to claim 1, wherein the method further comprises receiving one or more measured technical application properties for the one or more test polymers and determining based on the one or more measured technical application properties a plurality of new potential test synthesis specifications and/or new computed characterizing parameters and repeating the determination of one or more test synthesis specifications based on the new potential test synthesis specifications and/or new computed characterizing parameters.

6. An apparatus for providing one or more test synthesis specifications each associated with a test polymer for performing of an experiment series, wherein a test synthesis specification is suitable for synthesising the respective test polymer and measuring one or more technical application properties of the test polymer in the experiment series, wherein the apparatus comprises:
an input interface configured for:
a) receiving synthesis parameters defining ingredient and/or process parameter ranges in which one or more test polymers for the experiment series are to be determined, wherein different ingredient and/or process parameters are associated with different potential test polymers,
b) deriving a plurality of potential test synthesis specifications associated with respective potential test polymers based on the synthesis parameters,
c) providing computed characterizing parameters, wherein a computed characterizing parameter is indicative of a computed characteristic of a polymer and/or is derivable from one or more computed characteristics of the polymer,
one or more processors configured for:
a) determining for the provided computed characterizing parameters a computed characterizing parameter value for the potential test polymers based on the plurality of potential test synthesis specifications,
b) determining one or more test synthesis specifications associated with respective test polymers from the plurality of potential test synthesis specifications based on the determined computed characterizing parameter values, and
an output interface configured for providing the one or more test synthesis specifications comprising instructions for controlling a synthesis of the respective one or more test polymers.

7. An interface method for providing one or more test synthesis specifications each associated with a test polymer for performing of an experiment series, wherein the interface method comprises:
receiving, via an input unit, synthesis parameters defining ingredient and/or process parameter ranges in which one or more test polymers for the experiment series are to be determined,
interfacing, via an interface unit, with a processor performing the method according to claim 1 for providing the one or more test synthesis specifications comprising instructions for controlling a synthesis of the respective one or more test polymers, and
providing, via an output unit, the one or more test synthesis specifications.

8. A computer implemented method for generating a machine learning based determination model, wherein the trained determination model is adapted to determine a technical application property of a polymer based on one or more polymer computed characterizing parameters, wherein a polymer computed characterizing parameter is indicative of a computed characteristic of a polymer and/or is derivable from one or more computed characteristics of the polymer, wherein the method comprises:
receiving model synthesis parameters defining ingredient and/or process parameter ranges in which at least two training polymers for the training process are to be determined, wherein different ingredient and/or process parameters are associated with different potential training polymers,
deriving a plurality of potential training synthesis specifications associated with respective potential training polymers based on the model synthesis parameters,
providing computed characterizing parameters,
determining for the provided computed characterizing parameters a computed characterizing parameter value for the potential training polymers based on the plurality of potential training synthesis specifications,
determining at least two training synthesis specifications associated with respective training polymers from the plurality of potential training synthesis specifications based on the determined computed characterizing parameter values,
receiving for the at least two training polymers measured technical application property values of the technical application property, and
training the machine learning based determination model by parameterizing the determination model based on computed characterizing parameter values and the measured technical application property values of the at least two training polymers, and
providing the trained determination model.

9. A training apparatus for generating a machine learning based determination model, wherein the trained determination model is adapted to determine a technical application property of a polymer based on one or more polymer computed characterizing parameters, wherein a polymer computed characterizing parameter is indicative of a computed characteristic of a polymer and/or is derivable from one or more computed characteristics of the polymer, wherein the training apparatus comprises:
an input interface configured for
a) receiving model synthesis parameters defining ingredient and/or process parameter ranges in which at least two training polymers for the training process are to be determined, wherein different ingredient and/or process parameters are associated with different potential training polymers,
b) deriving a plurality of potential training synthesis specifications associated with respective potential training polymers based on the model synthesis parameters,
c) providing computed characterizing parameters,
one or more processors configured for:
a) determining for the provided computed characterizing parameters a computed characterizing parameter value for the potential training polymers based on the plurality of potential training synthesis specifications,
b) determining at least two training synthesis specifications associated with respective training polymers from the plurality of potential training synthesis specifications based on the determined computed characterizing parameter values,
c) receiving for the at least two training polymers measured technical application property values of the technical application property, and
d) training the machine learning based determination model by parameterizing the determination model based on the computed characterizing parameter values and the measured technical application property values of the at least two training polymers, and
an output interface configured for providing the trained determination model.

10. A computer-implemented method for controlling of an experiment series for determining a target polymer comprising a predetermined target technical application property, wherein the method comprises:
receiving a target value for the target technical application property,
receiving synthesis parameters defining ingredient and/or process parameter ranges in which the target polymer is to be searched, wherein different ingredient and/or process parameters are associated with different potential target polymers,
deriving a plurality of potential test synthesis specifications associated with respective potential test polymers based on the synthesis parameters,
providing computed characterizing parameters, wherein a computed characterizing parameter is indicative of a computed characteristic of a polymer and/or is derivable from one or more computed characteristics of the polymer
determining for the provided computed characterizing parameters a computed characterizing parameter value for the potential test polymers based on the plurality of potential test synthesis specifications,
determining one or more test synthesis specification associated with respective test polymers from the plurality of potential test synthesis specifications based on the determined computed characterizing parameter values,
generating control data for controlling the experiment series based on the one or more test synthesis specifications, wherein the control data comprise instructions for controlling a synthesis of the respective one or more test polymers and the performing of the respective measurement of the target technical application property,
receiving measured values for the target technical application properties for the one or more test polymers,
comparing the measured values of the target technical application property of the one or more test polymers with the target value and, based on the comparison, either i) determining a test polymer of the one or more test polymers as the target polymer and the respective test synthesis specification as the target synthesis specification, or ii) deriving a plurality of new potential test synthesis specifications and/or new computed characterizing parameters and repeating the determination of one or more test polymers and the measuring of the target technical application property utilizing the new potential test synthesis specification of the new potential target polymer, and
providing the determined target polymer and the target synthesis specification.

11. A determination apparatus for controlling of an experiment series for determining a target polymer comprising a predetermined target technical application property, wherein the apparatus comprises:
an input interface configured for:
a) receiving a target value for the target technical application property,
b) receiving synthesis parameters defining the ingredient and/or process parameter ranges in which the target polymer is to be searched, wherein different ingredient and/or process parameters are associated with different potential target polymers,
c) deriving a plurality of potential test synthesis specifications associated with respective potential test polymers based on the synthesis parameters,
d) providing computed characterizing parameters, wherein a computed characterizing parameter is indicative of a computed characteristic of a polymer and/or is derivable from one or more computed characteristics of the polymer, one or more processors configured for:
a) determining for the provided computed characterizing parameters a computed characterizing parameter value for the potential test polymers based on the plurality of potential test synthesis specifications,
b) determining one or more test synthesis specification associated with respective test polymers from the plurality of potential test synthesis specifications based on the determined computed characterizing parameter values,
c) generating control data for controlling the experiment series based on the one or more test synthesis specifications, wherein the control data comprise instructions for controlling a synthesis of the respective one or more test polymers and the performing of the respective measurement of the target technical application property,
d) receiving measured values for the target technical application properties for the one or more test polymers,
comparing the measured values of the target technical application property of the one or more test polymers with the target value and, based on the comparison, either i) determining a test polymer of the one or more test polymers as the target polymer and the respective test synthesis specification as the target synthesis specification, or ii) deriving a plurality of new potential test synthesis specifications and/or new computed characterizing parameters and repeating the determination of one or more test polymers and the measuring of the target technical application property utilizing the new potential test synthesis specification of the new potential target polymer, and
an output interface configured for providing the determined target polymer and the target synthesis specification.

12. A computer program product for providing one or more test synthesis specifications, wherein the computer program product comprises program code means for causing a computing system to execute the method according to claim 1.

13. Control data for controlling an experiment generated utilizing the method of claim 4.

14. A synthesis specification comprising instructions for controlling a synthesis of the respective one or more test polymers provided by a method according to claim 1.

15. Use of control data generated utilizing the method of claim 4 for controlling a synthesis system, in particular, laboratory equipment, for producing the one or more test polymers in accordance with the one or more test synthesis specifications.
